(19) United States
(12) Patent Application Publication
(10) Pub. No.: US 2006/0010513 A1
(43) Pub. Date: Jan. 12, 2006

(54) Oligonucleotide arrays to monitor gene expression and methods for making and using same

(76) Inventors: Mark W. Melville, Melrose, MA (US); Timothy S. Charlebois, Andover, MA (US); William M. Mounts, Andover, MA (US); Louane E. Hann, Boston, MA (US); Martin S. Sinacore, Andover, MA (US); Mark W. Leonard, Manchester, NH (US); Eugene L. Brown, Newton Highlands, MA (US); Christopher P. Miller, Arlington, MA (US); Gene W. Lee, Chelmsford, MA (US)

Correspondence Address: FITZPATRICK CELLA (WYETH), 30 ROCKEFELLER PLAZA, NEW YORK, NY, 10112-3800, US

(21) Appl. No.: 11/128,049

(22) Filed: May 11, 2005

Related U.S. Application Data
(60) Provisional application No. 60/570,425, filed on May 11, 2004.

(51) Int. Cl. A01H 1/00; C12N 15/82

(52) U.S. Cl. 800/278

(57) ABSTRACT

The present invention provides an oligonucleotide array capable of identifying genes and related pathways involved with the induction of a particular phenotype by a cell line, e.g., the genes and related pathways involved with the induction of transgene expression by the cell line. The invention is particularly useful when there is little or no information about the genome of the cell line being studied, because it provides methods for identifying consensus sequences for known and previously undiscovered genes, and for designing oligonucleotide probes to the identified consensus sequences. Additionally, when the array is to be used to determine optimal conditions for expression of a transgene by the cell line, the invention teaches methods of including oligonucleotide probes to transgene sequences in the array. The invention also provides methods of using the array to identify genes and related pathways involved with the induction of a particular cell line phenotype. The invention also provides novel polynucleotides of undiscovered genes (i.e., a gene that had not been sequenced and/or shown to be expressed by CHO cells) and novel polynucleotides involved with the induction of a particular cell phenotype, e.g., increased survival when grown under stressful culture conditions, increased transgene expression, decreased production of an antigen, etc. These novel polynucleotides are termed novel CHO sequences and differential CHO sequences, respectively. The invention also provides genetically engineered expression vectors, host cells, and transgenic animals comprising the novel nucleic acid molecules of the invention. The invention additionally provides antisense and RNAi molecules to the nucleic acid molecules of the invention. The invention further provides methods of using the polynucleotides of the invention.

This application claims the benefit of U.S. Provisional Application Ser. No. 60/570,425, filed May 11, 2004, incorporated herein by reference in its entirety.

This application incorporates by reference all materials on the compact discs labeled “Copy 1” and “Copy 2.” Each of the compact discs includes the following files: Table 2.txt (3,230 KB, created 11 May 2005), Table 2v2.txt (429 KB, created 11 May 2005), Table 3.txt (77.1 KB, created on 11 May 2005), Table 3v2.txt (7.82 KB, created on 11 May 2005), Table 4.txt (90.6 KB, created on 11 May 2005), Table 4v2.txt (3.93 KB, created on 11 May 2005), Table 5.txt (2,260 KB, created on 11 May 2005), Table 5v2.txt (425 KB, created on 11 May 2005), and “Sequence Listing” 01997027700.ST25.txt (7,150 KB, created on 11 May 2005). This application also incorporates by reference all materials on the compact disc labeled “CRF”; the compact disc includes “Sequence Listing” 01997027700.ST25.txt (7,150 KB, created on 11 May 2005).

### BACKGROUND OF THE INVENTION

1. Field of the Invention

The present invention is directed toward 1) methods of forming an oligonucleotide array for monitoring (e.g., detecting the absence, presence, or quantity of) the expression levels of genes, including previously undiscovered genes, of a cell, 2) methods of using the array to verify expression by a cell of previously undiscovered genes and to discover genes and related pathways that are involved in conferring a particular cell phenotype, e.g., that can be used in the optimization of cell line culture conditions and transgene expression, and 3) sequences involved in conferring a cell phenotype optimal for transgene expression.

2. Related Background Art

Fundamental to the present-day study of biology is the ability to optimally culture and maintain cell lines. Cell lines not only provide an in vitro model for the study of biological systems and diseases, but are also used to produce organic reagents. Of particular importance is the use of genetically engineered prokaryotic or eukaryotic cell lines to generate mass quantities of recombinant proteins. A recombinant protein may be used in a biological study, or as a therapeutic compound for treating a particular ailment or disease.

The production of recombinant proteins for biopharmaceutical application typically requires vast numbers of cells and/or particular cell culture conditions that influence cell growth and/or expression. In some cases, production of recombinant proteins benefits from the introduction of chemical inducing agents (such as sodium butyrate or valeric acid) to the cell culture medium. Identifying the genes and related genetic pathways that respond to the culture conditions (or particular agents) that increase transgene expression may elucidate potential targets that can be manipulated to increase recombinant protein production and/or influence cell growth.

Research into optimizing recombinant protein production has been primarily devoted to examining gene regulation, cellular responses, cellular metabolism, and pathways activated in response to unfolded proteins. Currently, there is no available method that allows for the simultaneous monitoring of transgene expression and identification of the genetic pathways involved in transgene expression. For example, currently available methods for detecting transgene expression include those that measure only the presence and amount of known proteins (e.g., Western blot analysis, enzyme-linked immunosorbent assay, and fluorescence-activated cell sorting), or the presence and amount of known messenger RNA (mRNA) transcripts (e.g., Northern blot analysis and reverse transcription-polymerase chain reaction). These and similar methods are not only limited in the number of known proteins and/or mRNA transcripts that can be detected at one time, but they also require that the investigator know or “guess” what genes are involved in transgene expression prior to experimentation (so that the appropriate antibodies or oligonucleotide probes are used). Another limitation inherent in blot analyses and similar protocols is that proteins or MRNA that are the same size cannot be distinguished. Considering the vast number of genes contained within a single genome, identification of even a minority of genes involved in a genetic pathway using the methods described above is costly and time-consuming. Additionally, the requirement that the investigator have some idea regarding which genes are involved does not allow for the identification of genes and related pathways that were either previously undiscovered or unknown to be involved in the regulation of transgene expression.

To overcome the limited number of transcripts that can be detected with hybridization protocols similar to Northern blot analysis, U.S. Pat. No. 6,040,138 provides a method of monitoring the expression of a multiplicity of genes using hybridization to oligonucleotide arrays, e.g., high-density oligonucleotide arrays (or microarrays). Hybridization to high-density oligonucleotide arrays provides a fast and reliable method to determine the presence and amount of known mRNA transcripts and can be readily applied in detecting diseases, identifying differential gene expression between two samples, and screening for compositions that upregulate or downregulate the expression of particular genes. Additionally, U.S. Pat. No. 6,040,138 teaches methods of optimizing oligonucleotide probesets to be included in the array.

However, the method described in U.S. Pat. No. 6,040,138 requires that oligonucleotide probes be made to the polynucleotide sequences of known genes. Consequently, the methods of making and using an array directed toward an organism as described in U.S. Pat. No. 6,040,138 cannot be used to detect the expression of previously undiscovered genes of a cell or cell line, i.e., genes that have not been previously sequenced and/or previously shown to be expressed by the particular cell line derived from an organism to which the array is directed. In other words, high density oligonucleotide arrays have not been directed toward, and thus have not been useful for, monitoring gene expression levels in cells or cell lines derived from an organism for which little genomic information is available (i.e., an unsequenced organism, e.g., monkeys, pigs, hamsters, etc.) (see, e.g., Korke et al. (2002) J. Biotech. 94:73-92). Monitoring the gene expression levels of such cells or cell lines has been performed using high-density oligonucleotide arrays directed toward other organisms for which the whole genome is available (or the sequencing effort is near completion) and that are phylogenetically close, e.g., use of human arrays to monitor gene expression levels in monkey cells (Gagneux and Varki (2001) Mol. Phylogenet. Evol. 18:2-13) and use of rodent arrays to monitor gene expression levels in cells derived from hamsters (Korke et al., supra). Additionally, the method described in U.S. Pat. No. 6,040,138 does not disclose a protocol with which sequences or subsequences (i.e., consecutive nucleotides identical to, but less than, the full sequence) of unknown genes can be determined. Consequently, whereas U.S. Pat. No. 6,040,138 allows for simultaneous monitoring of a multiplicity of genes, it does not solve the problem of identifying previously undiscovered genes and related genetic pathways, e.g., those that may be regulated in a cell in response to a particular culture condition. Additionally, U.S. Pat. No. 6,040,138 does not teach the use of microarray technology to either confirm or improve transgene expression by genetically engineered cells.

The present invention solves these problems by providing methods that will generate the sequences and subsequences of previously undiscovered genes in a cell or cell line, e.g., cells or cell lines derived from unsequenced organisms. The invention also provides a method by which these sequences are used to generate an oligonucleotide array that may be used to 1) verify expression of previously undiscovered genes, 2) verify expression of a transgene, and 3) determine genes (including previously undiscovered genes) and related genetic pathways that are involved (directly or indirectly) with a particular cell phenotype, e.g., increased and efficient transgene expression. Discovery of these genes and/or related pathways will provide new targets that can be manipulated to improve the yield and quality of recombinant proteins and influence cell growth.

### SUMMARY OF THE INVENTION

The present invention utilizes oligonucleotide microarray technology to identify genes and related pathways regulated in response to specific culture conditions, especially those conditions that result in optimal expression of transferred genes (transgenes) by genetically engineered cells or genetically engineered cell lines. In particular, the invention provides methods for forming an oligonucleotide array directed toward unsequenced organisms, which methods generally comprise determining the sequences or subsequences of genes expressed by the cell line, and designing an oligonucleotide array for these sequences. The sequences or subsequences of genes expressed by the cell line are determined by collecting a plurality of nucleic acid sequences, clustering and aligning said plurality of nucleic acid sequences, and identifying consensus sequences from the clustered and aligned plurality of nucleic acid sequences. Oligonucleotide probes are then designed based on identified consensus sequences, as well as transgene and control sequences. The oligonucleotide probes may then be immobilized in a random but known location on a surface to form the oligonucleotide array.

Thus the invention provides a method of forming an oligonucleotide array directed toward an unsequenced organism, wherein the method comprises the steps of (1) identifying a plurality of template sequences, wherein the plurality comprises at least one consensus sequence for a gene expressed by the unsequenced organism, and (2) selecting a plurality of oligonucleotide probes, wherein the plurality of oligonucleotide probes comprises a first set of oligonucleotide probes, each of which is specific for one of the plurality of template sequences, and wherein at least one oligonucleotide probe is specific for the at least one consensus sequence for a gene expressed by a cell derived from the unsequenced organism; wherein the step of selecting the plurality of oligonucleotide probes forms the oligonucleotide array. In one embodiment of the invention, the at least one consensus sequence for the unsequenced organism may be generated from at least two nucleic acid sequences of different genera of the unsequenced organism, and/or from at least two nucleic acid sequences of different species of the unsequenced organism. For example, the unsequenced organism may be hamster, and the consensus sequence may be generated from a nucleic acid sequence of a cell derived from, e.g., Mesocrecetus auratus (Golden Hamster) and a nucleic acid sequence of a cell derived from, e.g., Cricetulus migratorius (Armenian Hamster). Alternatively, the consensus sequence may be generated from a nucleic acid sequence of a cell derived from, e.g., Cricetulus migratorius (Armenian Hamster) and a nucleic acid sequence of a cell derived from, e.g., Cricetulus griseus (Chinese Hamster). In some embodiments, the plurality of template sequences comprises at least one template sequence selected from the group consisting of the polynucleotide sequences of SEQ ID NOs:19-3572 and SEQ ID NOs:3661-7214, complements thereof, and subsequences thereof. In other embodiments, the plurality of template sequences may further comprise at least one other hamster sequence (e.g., a hamster sequence selected from the group consisting of the polynucleotide sequences of SEQ ID NOs:3573-3575 and SEQ ID NOs:7215-7217, complements thereof, and subsequences thereof), at least one transgene sequence (e.g., a transgene sequence selected from the group consisting of the polynucleotide sequences of SEQ ID NOs:1-18 and SEQ ID NOs:3643-3660, complements thereof, and subsequences thereof) and/or at least one control sequence (e.g., a control sequence selected from the group consisting of the polynucleotide sequences of SEQ ID NOs:3576-3642 and SEQ ID NOs:7218-7284, complements thereof, and subsequences thereof). Also, the plurality of oligonucleotide probes may further comprise a second set of oligonucleotide probes, each of which is a mismatch probe for a different oligonucleotide probe. The method of forming an oligonucleotide array may also include a last step of immobilizing the plurality of oligonucleotide probes to a solid phase support.

The invention also provides oligonucleotide arrays (that may or may not be immobilized to a solid phase support) directed toward an unsequenced organism. Generally, such arrays comprise a first plurality of oligonucleotide probes, each of which is specific to one of a plurality of template sequences, wherein the plurality of template sequences comprises at least one consensus sequence for a gene expressed by a cell derived from the unsequenced organism. In one embodiment of the invention, the consensus sequence may be generated from at least two nucleic acid sequences of different genera of the unsequenced organism, and/or from at least two nucleic acid sequences of different species of the unsequenced organism. In some embodiments, the plurality of template sequences comprises a template sequence selected from the group consisting of the polynucleotide sequences of SEQ ID NOs:19-3572 and SEQ ID NOs:3661-7214, complements thereof, and subsequences thereof. In other embodiments, the plurality of template sequences may further comprise at least one other hamster sequence (e.g., a hamster sequence selected from the group consisting of the polynucleotide sequences of SEQ ID NOs:3573-3575 and SEQ ID NOs:7215-7217, complements thereof, and subsequences thereof), at least one transgene sequence (e.g., a transgene sequence selected from the group consisting of the polynucleotide sequences of SEQ ID NOs:1-18 and SEQ ID NOs:3643-3660, complements thereof, and subsequences thereof) and/or at least one control sequence (e.g., a control sequence selected from the group consisting of the polynucleotide sequences of SEQ ID NOs:3576-3642 and SEQ ID NOs:7218-7284, complements thereof, and subsequences thereof). Also, the array may further comprise a second plurality of oligonucleotide probes, each of which is a mismatch probe for a different oligonucleotide probe.

It will be clear to one of skill in the art that the present invention is particularly useful for a cell line when both known genes and previously undiscovered genes (i.e., genes that, at the time of experimentation, have not been sequenced, or were sequenced but not shown to be expressed by the cell line) are included in said plurality of nucleic acid sequences. Generally, the nucleic acid sequences of known genes will be available from public databases. In contrast, the nucleic acid sequences of previously undiscovered genes must be obtained using other methods, such as generating a complementary DNA (cDNA) library for the cell line and identifying expressed sequence tags from the library. It is part of the present invention to provide nucleic acid sequences of previously undiscovered genes such that the oligonucleotide probes specific for the sequences (or subsequences thereof) of previously undiscovered genes may be included on an oligonucleotide array, and such that, via methods of using the oligonucleotide array, expression of such previously undiscovered genes by the cell line may be determined and/or verified to be involved in conferring a particular cell phenotype.

The invention is also related to methods of using the array, generally comprising the steps of providing a pool of target nucleic acids comprising, or derived from, MRNA transcripts isolated from a sample of the cell line; incubating the pool of target nucleic acids with the oligonucleotide array to allow target nucleic acids to hybridize to complementary oligonucleotide probes; and detecting the hybridization profile resulting from the target nucleic acids hybridizing with the corresponding complementary oligonucleotide probes. The invention comprises analyzing the resulting hybridization profile for useful information; for example, the analysis of the hybridization profile will yield information regarding the genes and related pathways activated during a particular culture condition that influences the expression of a particular cell phenotype.

Thus, the invention provides methods for detecting the absence, presence, and/or quantity of expression levels of a plurality of genes in a cell derived from an unsequenced organism. These methods generally comprise forming a hybridization profile by incubating target nucleic acids prepared from a cell with an array of the invention, and detecting the hybridization profile, wherein the hybridization profile is indicative of the absence, presence, and/or quantity of expression levels of a plurality of genes in the cell. As described above, the method may be particularly useful for detecting the absence, presence, and/or quantity of expression level of a previously undiscovered gene of the cell and/or a transgene. In some embodiments, the unsequenced organism is a hamster. In other embodiments, the cell is a CHO cell.

The invention also provides a method for comparing expression levels of a plurality of genes in a first cell derived from an unsequenced organism to expression levels of the plurality of genes in a second cell derived from the unsequenced organism, the method comprising the steps of (a) forming a first and second hybridization profile, wherein the first hybridization profile is formed by incubating target nucleic acids prepared from the first cell with a first array of the invention, and wherein the second hybridization profile is formed by incubating target nucleic acids prepared from the second cell with a second array identical to the first array; (b) detecting the first and second hybridization profiles; and (c) comparing the first and second hybridization profiles. In one embodiment of the invention, the first cell and the second cell are from the same cell line, wherein the first cell is modified with a transgene, and wherein the second cell is not modified with the transgene. In another embodiment, the first cell differs from the second cell with respect to a culture condition, e.g., duration of culture, temperature, serum concentration, nutrient concentration, metabolite concentration, pH, lactate concentration, ammonia concentration, oxidation level, sodium butyrate concentration, valeric acid concentration, hexamethylene bisacetamide concentration, cell concentration, cell viability, and recombinant protein concentration.

In another preferred embodiment of the invention, information related to gene expression levels aids in the diagnosis and remedy of suboptimal culture conditions, and/or in determining whether a cell line has been successfully engineered to express a transgene. One of skill in the art will recognize that such information can be particularly useful in optimizing transgene expression by various cell lines.

As such, the invention also provides isolated polynucleotides that are of previously undiscovered genes and/or are involved with the survival of cells when grown under stressful conditions, transgene expression, and/or the production of potential antigens, and methods of using polynucleotides of the invention to identify compounds capable of increasing transgene expression by a cell population. An isolated polynucleotide of the invention may have a polynucleotide sequence selected from the group consisting of the polynucleotide sequences of SEQ ID NOs:3421-3574, complements thereof, and subsequences thereof (e.g., a polynucleotide sequence selected from the group consisting of the polynucleotide sequences of SEQ ID NOs:7063-7216, complements thereof, and subsequences thereof). The invention also provides genetically engineered expression vectors, host cells, and transgenic animals comprising the nucleic acid molecules of the invention. The invention additionally provides inhibitory polynucleotides, e.g., antisense and RNA interference (RNAi) molecules, to the nucleic acid molecules of the invention. The invention further provides methods of using inhibitory polynucleotides of the invention to increase transgene expression by a population of cells, e.g., CHO cells.

### BRIEF DESCRIPTION OF THE DRAWINGS

FIG. 1: Generation of a Consensus Sequence and Complementary Oligonucleotide Probes for a Multi-Sequence Cluster

The GenBank sequence designated Accession No. AB014876, subject to clustering and alignment analysis, formed a multi-sequence cluster with two expressed sequence tag (EST) sequences obtained from a Chinese Hamster Ovary (CHO) cDNA library. Regions of low-complexity sequence and vector sequence were replaced with X's (boxed regions), and the unambiguous and consecutive homologous regions were used as templates to generate perfect match oligonucleotide probes 25 nucleotides in length. Examples of such probes are shown in the figure, which presents nucleotides 1-300 of the full-length GenBank sequence (i.e., 709 nucleotides).

### DETAILED DESCRIPTION OF THE INVENTION

The invention disclosed herein is directed toward an oligonucleotide array that can be used to verify the expression of a plurality of genes (including previously undiscovered genes) by a cell (or cell line) derived from an unsequenced organism, and to identify genes (including previously undiscovered genes) and related pathways that may be involved with the induction of a particular cell phenotype, e.g., increased and efficient transgene expression. Thus the invention provides the arrays, methods of making such arrays, and methods of using such arrays to 1) monitor (e.g., detect the absence, presence, and/or quantity of) expression levels of a plurality of genes, including previously undiscovered genes and/or transgenes, by a cell or cell line, and/or 2) determine genes and related pathways involved with conferring a particular cell phenotype, e.g., increased transgene expression, the methods comprising the steps of using an array of the invention. Accordingly, the present invention also provides sequences that are shown to be involved in transgene regulation, some of which are previously undiscovered genes, i.e., genes that, at the time of experimentation, had not been sequenced, or were sequenced but not verified to be expressed by the cell line.

Method of Making and Array of the Invention

An object of the present invention is to provide a method of forming an oligonucleotide array that can be used to verify the expression of previously undiscovered genes by a cell (e.g., a cell line) and to identify genes (including previously undiscovered genes) and related pathways that may be involved with the induction of a particular cell phenotype, e.g., increased and/or efficient transgene expression. As taught herein, the method of forming an oligonucleotide array directed toward an unsequenced organism comprises the steps of (1) identifying a plurality of template sequences, wherein the plurality comprises at least one consensus sequence for a gene expressed by a cell derived from the unsequenced organism, and (2) selecting a plurality of oligonucleotide probes, wherein the plurality of oligonucleotide probes comprises a first set of oligonucleotide probes, each of which is specific for one of the plurality of template sequences, and wherein at least one oligonucleotide probe is specific for the at least one consensus sequence for a gene expressed by the unsequenced organism; wherein the step of selecting the plurality of oligonucleotide probes forms the array of nucleic acids.

I) Identification of Template Sequences

Template sequences are those sequences to which oligonucleotide probes of the invention will hybridize under oligonucleotide array hybridization conditions. Additionally, a template sequence may be a consensus sequence to a gene of a cell (including a previously unidentified gene), a transgene sequence, or a control sequence. The identification of consensus sequences to known or previously undiscovered genes of a cell derived from an unsequenced organism is described.

The consensus sequences are identified by the well-known method of clustering and aligning a plurality of nucleic acid sequences. Nucleic acid sequences may be gene coding sequences and/or expressed sequence tag (EST) sequences.

Whether gene coding sequences are open reading frame (ORF) sequences or exon sequences, which may include 5′ or 3′ untranslated regions (UTRs) in addition to the ORF sequence, depends on the source organism from which the gene coding sequences are obtained. For example, if the source organism is prokaryotic, gene coding sequences are single-exon ORF sequences that do not contain 5′ or 3′ UTRs. However, if the source organism is eukaryotic, the gene coding sequences are comprised of multiple exon sequences, which may include 5′ or 3′ UTRs. As protocols used in the invention, such as the in vitro transcription protocol, are 3′-biased (based on the utilization of the oligo-dT primer), exon sequences, specifically those containing 3′ UTR sequence, as opposed to simply the ORF sequences, should be used whenever possible. However, if these transcription protocols are replaced with unbiased protocols, the inclusion of 3′ UTRs becomes less important. For the sake of clarity, use of the phrase “gene coding sequence” includes ORF and/or exon sequences, whichever is appropriate according to source organism and transcription protocols of the invention.

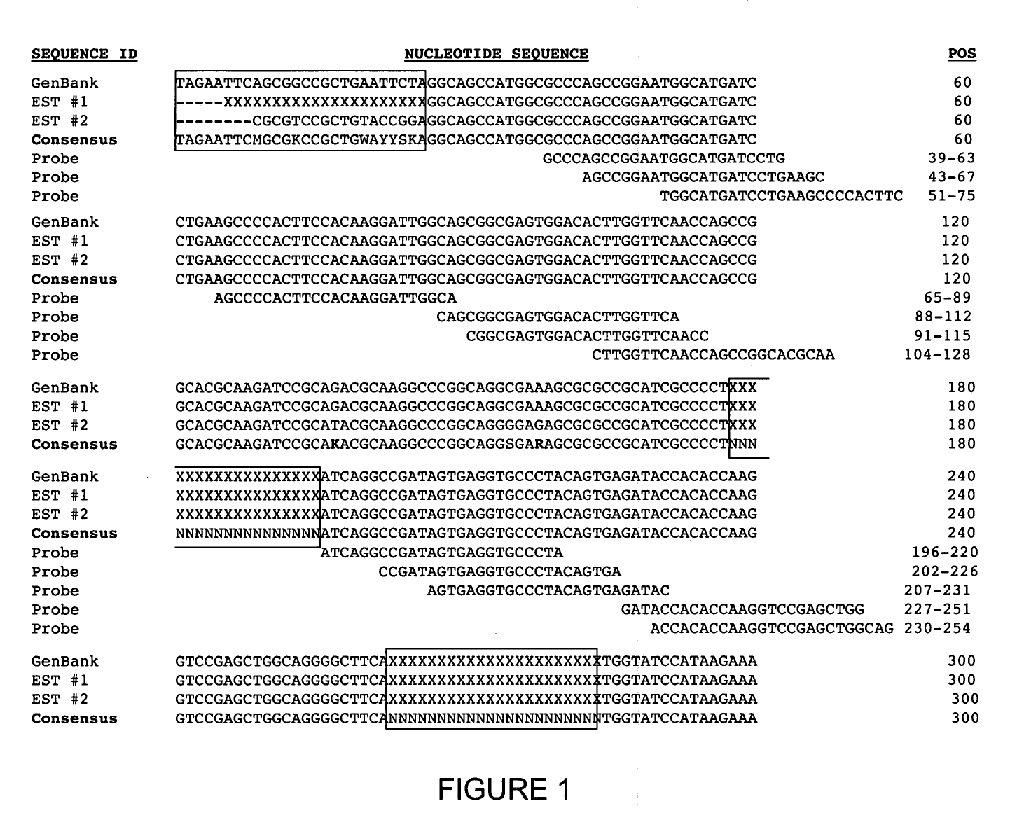

Preferred gene coding sequences of the invention may be obtained from incomplete and complete genomic sequences that are publicly available, or may be generated by prediction algorithms that are well known in the art. For example, gene coding sequences that are generated by prediction algorithms may include previously undiscovered genes. In a preferred embodiment, when both incomplete and complete genomic sequences are used, the incomplete genomes are oriented based on alignment to complete genomes. Additionally, gene coding sequences are separated based on whether they are oriented 5′ to 3′ on the sense (plus) strand or the antisense (minus) strand of their respective genome prior to clustering and alignment, such that plus and minus gene coding sequences are analyzed separately. Separately analyzing of the plus or minus gene coding sequences prevents the clustering and alignment of gene coding sequences that overlap each other on opposite strands of the genomic sequence. Although the strand assignment is arbitrary, it may be performed such that the genomic sequences that provided the gene coding sequences are highly conserved in primary and secondary structure. For example, upon orienting the genomic sequences, sequence fragments for each incomplete genome can be bridged with six-frame stop sequences, an example of which is 5′-CTAACTAATTAG-3′ (set forth as SEQ ID NO:7285). The plus or minus assignment then proceeds such that gene coding sequences obtained from incomplete genomes are assigned the same designation as highly homologous or identical regions on complete genomes. In another preferred embodiment, the genomic sequences are also screened for low-complexity sequence regions (repeats, etc.) and contaminating vector sequences. Any stretch of a genomic sequence meeting these criteria may be masked by replacing the nucleotides with a poly-X sequence of similar length prior to clustering and aligning. Three examples of such poly-X sequences are shown in FIG. 1.

One of skill in the art will recognize that it will be easier to assign gene coding sequences to the plus or minus strand when differences among the genomic sequences are small. In other words, the orientation of incomplete genomic sequences to complete genomic sequences will be easier when, e.g., the genomic sequences are obtained from different strains of a bacterial species as compared to when, e.g., the genomic sequences are obtained from different species and/or genera of an animal. Although strand assignment of the gene coding sequences may not be possible, e.g., when they are obtained from different species and/or genera of an animal, the lack of gene coding sequence separation will not affect the invention, as separation of gene coding sequences prior to alignment and clustering is just one embodiment of the invention.

Preferred EST sequences of the invention may be obtained from cDNA libraries generated from cells or cell lines using methods well known in the art; such methods are exemplified in Example 1.1. One of skill in the art will recognize that including EST sequences obtained from cells or cell lines grown in different culture conditions will increase the potential of including sequences of genes involved in, e.g., cell growth and maintenance and/or transgene production. A skilled artisan will also recognize that EST sequences generated from a cDNA library are generally submitted in a 3′ to 5′ direction. In one embodiment of the invention, an internal 3′ read, e.g., a poly-T tail, is included in all EST sequences. This internal 3′ read provides quality assurance regarding the directionality of the EST sequence (e.g., whether the sequence is disclosed 3′ to 5′, or vice versa). Additionally, the 3′ read provides a means by which to orient a consensus sequence identified from the EST sequence. When necessary, suspicious EST sequences, e.g., those for which orientation is unknown and/or may not be inferred from other sequences in the sequence collection, may be excluded from the cluster and alignment analysis. Alternatively, it may be beneficial to include the reverse complement of the suspicious sequence in the initial cluster and alignment analysis.

It also will be apparent to one of skill in the art that, whereas any gene coding sequence and/or EST sequence may be used, the most useful nucleic acid sequences are isolated from either the cells or cell line(s) to be monitored or the unsequenced organism (e.g., unsequenced animal) from which the cell line was derived. Gene coding sequences and EST sequences of the animal from which the cell line was derived can be isolated from any genus, species or strain that has the same animal classification. As a nonlimiting example, when culturing and monitoring the Chinese Hamster Ovary (CHO) cell line derived from the hamster (an unsequenced organism), gene coding sequences and EST sequences isolated from CHO cells, Cricetulus griseus (Chinese hamster), as well as other hamsters, such as Cricetulus migratorius (Armenian hamster) and Mesocricetus auratus (Golden hamster), can be clustered and aligned to identify consensus sequences. A skilled artisan will recognize that inclusion of gene coding sequences and/or EST sequences from animals other than the genus and/or species from which the cell was derived increases the likelihood that a consensus sequence to a previously undiscovered gene of the cell will be identified.

Gene coding sequences and EST sequences are clustered such that homologous sequences (defined by parameters such as sequence identity over a certain number of base pairs), and single transcripts that were included in the plurality of nucleic acid sequences multiple times, may be aligned. Suitable clustering and alignment methods include, but are not limited to, manually curating the sequences, utilizing well-defined computer software packages, or a combination of both. In a preferred embodiment, clustering and alignment methods are repeated and the parameters that define homologous sequences become more stringent with each repetition of clustering and alignment. For example, one of skill in the art may begin the clustering and alignment method by defining homologous sequences as those that demonstrate a minimum threshold of 85% sequence identity over a 300 base pair region. In subsequent repetitions of clustering and alignment, the definition of homologous sequences may become more stringent, e.g., it may be defined as sequences that demonstrate 90% sequence identity over a 100 base pair region. Such parameters are well known to one of skill in the art. In a more preferred embodiment of the invention, all clusters are manually curated to verify cluster membership. Upon manual curation, and prior to the identification of consensus sequences, some clusters are joined or separated based on homologies well known in the art.

One of skill in the art will recognize that some of the methods by which the plurality of nucleic acid sequences is obtained may cause the gene coding sequences or EST sequences to contain regions that are not truly contained within the genomic sequences or cDNA sequences from which the gene coding sequences or EST sequences are derived. These regions may include, e.g., portions of the expression vectors used to sequence the gene coding sequences or EST sequences. As such, screening the plurality of nucleic acid sequences for these regions, and similar regions, e.g., low-complexity regions, prior to the clustering and alignment analysis will aid in clustering and aligning homologous gene coding sequences and/or EST sequences. One of skill in the art will recognize that masking vector regions or low-complexity regions will increase the likelihood that homologous sequences will cluster because they represent single transcripts included in the plurality of nucleic acid sequences multiple times, and not because they contain similar vector regions or low-complexity regions.

Consensus sequences are generated for singleton clusters containing an exemplar sequence (i.e., only one gene coding sequence or EST sequence), and multi-sequence clusters containing more than one gene coding sequence and/or EST sequence. The consensus sequence for a singleton cluster is simply the sequence of the exemplar sequence. However, a consensus sequence for a multi-sequence cluster is derived after aligning each of the sequences within a multi-sequence cluster, and identifying a consensus nucleotide for each position of the consensus sequence.

The consensus nucleotide at a particular position of the consensus sequence depends on the nucleotides present at the same position in the clustered and aligned sequences. If the nucleotides at a given position of the alignment are identical for each of the clustered and aligned sequences, then the resulting consensus nucleotide at that position is the nucleotide in common. However, if the nucleotides at a given position of the alignment are different among the clustered and aligned sequences, then the resulting consensus nucleotide at that position is designated with an ambiguous nucleotide code according to International Union of Pure and Applied Chemistry (IUPAC) base representation, which is consistent with the WIPO standard ST.25 (IUPAC-IUB Symbols For Nucleotide Nomenclature: Cornish-Bowden (1985) Nucl. Acids Res. 13:3021-30). These nucleotide differences may be due to variations in the sequences clustered in the multi-sequence clusters and/or the inability to distinguish the correct nucleotide for a particular position, i.e., areas of low homology. Regardless of the cause, nucleotide differences among clustered and aligned gene coding and/or EST sequences are not resolved in the consensus sequence; this prevents biasing probes towards one particular gene coding sequence and/or EST sequence. In other words, consensus sequences containing ambiguous nucleotides may still be used to generate oligonucleotide probes. During probe selection, as described in greater detail below, these areas of low homology are taken into account and oligonucleotides to these regions are excluded.

In addition to gene coding sequences and EST sequences (e.g., from cells or cell line(s) to be monitored, animals from which the cell line was derived, etc.), it will be clear to one of skill in the art that inclusion of transgene sequences in the alignment and clustering analysis will prove beneficial, especially when the array is used to determine the optimal conditions for expression of the transgene by a cell line. Transgene sequences can include product sequences that code for the recombinant protein of interest and product-related sequences that are often transferred with the product sequence, such as the gene for the resistance marker neomycin. When transgene sequences are included in the clustering and alignment analysis, it may be the case that they will cluster with consensus sequences of the cell line, even if the transgene sequence and cell line are from different animals. However, due to the disparity between gene sequences of different animals, a transgene sequence, or portions thereof, should align by itself. Again, manual curation of all multi-sequence clusters ensures proper sequence membership for all clustering and alignment results. Nonlimiting examples of exemplary transgene sequences are shown in Table 1.

TABLE 1
Exemplary transgenes

| Name | SEQ ID NO |
|---|---|
| Neomycin phosphotransferase II | 1 |
| Internal ribosomal entry site (IRES) | 2 |
| Human bone morphogenetic protein 2A | 3 |
| Hamster dihydrofolate reductase | 4 |
| Human beta-1,6-N-acetylglucosaminyltransferase | 5 |
| Human alpha(1,3)fucosyltransferase | 6 |
| Human antibody against A-beta protein (light chain) | 7 |
| Human antibody against A-beta protein (heavy chain) | 8 |
| Mouse dihydrofolate reductase | 9 |
| Human paired basic amino acid cleaving enzyme (PACE) | 10 |
| Human p-selectin glycoprotein ligand-1 | 11 |
| Human recombinant coagulation factor IX | 12 |
| Human recombinant coagulation factor VIII | 13 |
| (B-domain deleted) | |
| Human soluble interleukin-13 receptor, alpha 2 | 14 |
| Human blood platelet membrane glycoprotein IB-alpha | 15 |
| (N-terminus) | |
| fused to mutated Fc IgG1 | |
| Human soluble TNF receptor-2 p75 | 16 |
| Human antibody against myostatin (light chain) | 17 |
| Human antibody against myostatin (heavy chain) | 18 |

In one embodiment of the invention, publicly available and predicted gene coding sequences and EST sequences from hamsters (e.g., gene coding sequences and/or EST sequences from Mesocricetus auratus (Golden Hamster), Cricetulus migratorius (Armenian hamster), Cricetulus griseus (Chinese Hamster), the CHO cell line, etc.) are aligned to identify consensus sequences. Exemplary consensus sequences identified by clustering and aligning publicly available and predicted gene coding sequences and EST sequences from hamsters are listed in Table 2 and set forth as SEQ ID NOs:19-3572. Table 2 provides the SEQ ID NO of each listed sequence, an accession number for each listed sequence, the one or more species from which the consensus sequence was obtained, a header for each consensus sequence, wherein each header includes a qualifier as well as other information for the corresponding sequence, and the nucleotide sequence of each sequence. As demonstrated in Example 3 below, a plurality of the consensus sequences listed in Table 2 were previously undiscovered genes of CHO cells (i.e., have not been sequenced before or shown to be expressed in CHO cells) but the expression of which in CHO cells is now verified, and/or were not previously known to be involved in the survival of cells grown under stressful conditions, transgene expression, and/or production of possible antigens, but of which the downregulation is correlated with survival, increased transgene expression, and/or decreased production of possible antigens. Listed in Tables 2 and 3 and set forth as SEQ ID NOs: 3439-3573 are nonlimiting and exemplary gene sequences that were previously undiscovered but are verifiably expressed by CHO cells. Listed in Tables 2 and 4 and set forth as SEQ ID NOs:3421-3572 are nonlimiting and exemplary gene sequences demonstrated to be involved in cell survival when cells are cultured under stressful conditions, with increased transgene expression, and/or a lower production of the sialic acid N-glycolylneuraminic acid (NGNA); thus, these sequences may serve as exemplary targets to increase the survival of cells grown under stressful culture conditions, increase transgene expression by gene modified cells, and/or decrease the production of possible human antigens by cells. Also listed in Table 2 are other hamster sequences, i.e., hamster caspase 8, hamster caspase 9, and hamster BCLXL, which are set forth as SEQ ID NOs:3573-3575, respectively. Table 2 also provides a list of control sequences set forth as SEQ ID NOs:3576-3642.

II) Selecting Oligonucleotide Probes

Oligonucleotide probes used in this invention comprise nucleotide polymers or analogs and modified forms thereof such that hybridizing to a pool of target nucleic acids occurs in a sequence specific manner under oligonucleotide array hybridization conditions. As used herein, the term “oligonucleotide array hybridization conditions” refers to the temperature and ionic conditions that are normally used in oligonucleotide array hybridization. In many examples, these conditions include 16-hour hybridization at 45° C., followed by at least three 10-minute washes at room temperature. The hybridization buffer comprises 100 mM MES, 1 M [Na+], 20 mM EDTA, and 0.01% Tween 20. The pH of the hybridization buffer can range between 6.5 and 6.7. The wash buffer is 6×SSPET, which contains 0.9 M NaCl, 60 mM NaH2PO4, 6 mM EDTA, and 0.005% Triton X-100. Under more stringent oligonucleotide array hybridization conditions, the wash buffer can contain 100 mM MES, 0.1 M [Na+], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

As is known by one of skill in the art, oligonucleotide probes can be of any length. Preferably, oligonucleotide probes of the invention are 20 to 70 nucleotides in length. Most preferably, oligonucleotide probes of the invention are 25 nucleotides in length. In one embodiment, the nucleic acid probes of the present invention have relatively high sequence complexity. In many examples, the probes do not contain long stretches of the same nucleotide. In addition, the probes may be designed such that they do not have a high proportion of G or C residues at the 3′ ends. In another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Oligonucleotide probes are made to be specific for (e.g., complementary to (i.e., capable of hybridizing to)) a template sequence. Any part of a template sequence can be used to prepare probes. Multiple probes, e.g., 5, 10, 15, 20, 25, 30, or more, can be prepared for each template sequence. These multiple probes may or may not overlap each other. Overlap among different probes may be desirable in some assays. In many embodiments, the probes for a template sequence have low sequence identities with other template sequences, or the complements thereof. For instance, each probe for a template sequence can have no more than 70%, 60%, 50% or less sequence identity with other template sequences, or the complements thereof. This reduces the risk of undesired cross-hybridization. Sequence identity can be determined using methods known in the art. These methods include, but are not limited to, BLASTN, FASTA, and FASTDB. The Genetics Computer Group (GCG) program, which is a suite of programs including BLASTN and FASTA, can also be used. Preferable sequences for template sequences include, but are not limited to, consensus sequences, transgene sequences, and control sequences (i.e., sequences used to control or normalize for variation between experiments, samples, stringency requirements, and target nucleic acid preparations). Additionally, any subsequence of consensus, transgene and control sequences can be used as a template sequence. In one embodiment of the invention, at least one consensus sequence listed in Table 2 is used as a template sequence. In a preferred embodiment of the invention, at least one consensus sequence listed in Table 3 is used as a template sequence. In another preferred embodiment of the invention, at least one consensus sequence listed in Table 4 is used as a template sequence.

In one embodiment of the invention, only certain regions (i.e., tiling regions) of consensus, transgene and control sequences are used as template sequences for the oligonucleotide probes used in this invention. One of skill in the art will recognize that protocols that may be used in practicing the invention, i.e., in vitro transcription protocols, often result in a bias toward the 3′-ends of target nucleic acids. Consequently, in one embodiment of the invention, the region of the consensus sequence or transgene sequence closest to the 3′-end of a consensus sequence is most often used as a template for oligonucleotide probes. Generally, if a poly-A signal could be identified, the 1400 nucleotides immediately prior to the end of the consensus or transgene sequences are designated as a tiling region. Alternatively, if a poly-A signal could not be identified, only the last 600 nucleotides of the consensus or transgene sequence are designated as a tiling region. However, it should be noted that the invention is not limited to using only these tiling regions within the consensus, transgene and control sequences as templates for the oligonucleotide probes. Indeed, a tiling region may occur anywhere within the consensus, transgene or control sequences. For example, as described in greater detail below, the tiling region of a control sequence may comprise regions from both the 5′ and 3′-ends of the control sequence. In fact, the entire consensus, transgene or control sequence may be used as a template for oligonucleotide probes. Tiling sequences that may be used for each of the transgene sequences set forth in Table 1; and the consensus sequences, other hamster sequences, and control sequences set forth in Table 2; are listed in Table 5 and are set forth as SEQ ID NOs:3643-7284, where SEQ ID NO:3642+n is an exemplary tiling sequence for SEQ ID NO:n (e.g., SEQ ID NO:3643 may be used as the tiling sequence for SEQ ID NO:1; SEQ ID NO:3661 may be used as the tiling sequence for SEQ ID NO:19; SEQ ID NO:7213 may be used as the tiling sequence for SEQ ID NO:3571; etc.).

In one embodiment of the invention, an oligonucleotide array is designed to comprise perfect match probes to a plurality of consensus sequences (i.e., consensus sequences for multi-sequence clusters, and consensus sequences for exemplar sequences) identified as described above. In another embodiment, the oligonucleotide array is designed to comprise perfect match probes to both consensus and transgene sequences. It will be apparent to one of skill in the art that inclusion of oligonucleotide probes to transgene sequences will be useful when a cell line is genetically engineered to express a recombinant protein encoded by a transgene sequence, and the purpose of the analysis is to confirm expression of the transgene and determine the level of such expression. In those cases where the transgene is linked in a bicistronic mRNA to a downstream ORF, such as dihydrofolate reductase (DHFR), the level of transgene expression may also be determined from the level of expression of the downstream sequence. In another embodiment of the invention, the oligonucleotide array further comprises control probes that normalize the inherent variation between experiments, samples, stringency requirements, and preparations of target nucleic acids. The composition of each of these types of control probes is described in U.S. Pat. No. 6,040,138, incorporated herein in its entirety by reference. For a more detailed description, the purposes of the control probes are briefly described below.

It is well known to one of skill in the art that two pools of target nucleic acids individually processed from the same sample can hybridize to two separate but identical oligonucleotide arrays with varying results. The varying results between these arrays are attributed to several factors, such as the intensity of the labeled pool of target nucleic acids and incubation conditions. To control for these variations, normalization control probes can be added to the array. Normalization control probes are oligonucleotides exactly complementary to known nucleic acid sequences spiked into the pool of target nucleic acids. Any oligonucleotide sequence may serve as a normalization control probe; in a preferred embodiment, the normalization control probes are created from a template obtained from an organism other than that from which the cell line being analyzed is derived. In another preferred embodiment, an oligonucleotide array to mammalian sequences will contain normalization oligonucleotide probes to the following genes: bioB, bioC, and bioD from the organism Escherichia coli, cre from the organism Bacteriophage PI, and dap from the organism Bacillus subtilis, or subsequences thereof. The signal intensity received from the normalization control probes are then used to normalize the signal intensities from all other probes in the array. Additionally, when the known nucleic acid sequences are spiked into the pool of target nucleic acids at known and different concentrations for each transcript, a standard curve correlating signal intensity with transcript concentration can be generated, and expression levels for all transcripts represented on the array can be quantified (see, e.g., Hill et al. (2001) Genome Biol. 2(12):research0055.1-0055.13).

Due to the naturally differing metabolic states between cells, expression of specific target nucleic acids vary from sample to sample. In addition, target nucleic acids may be more prone to degradation in one pool compared to another pool. Consequently, in another embodiment of the invention, the oligonucleotide array further comprises oligonucleotide probes that are exactly complementary to constitutively expressed genes, or subsequences thereof, that reflect the metabolic state of a cell. Nonlimiting examples of these types of genes are beta-actin, transferrin receptor and glyceraldehyde-3-phosphate dehydrogenase (GAPDH).

In one embodiment of the invention, the pool of target nucleic acids is derived by converting total RNA isolated from the sample into double-stranded cDNA and transcribing the resulting cDNA into complementary RNA (cRNA) using methods described in more detail in the Examples. The RNA conversion protocol is started at the 3′-end of the RNA transcript, and if the process is not allowed to go to completion (if, for example, the RNA is nicked, etc.) the amount of the 3′-end message compared to the 5′-end message will be greater, resulting in a 3′-bias. Additionally, RNA degradation may start at the 5′-end (Jacobs Anderson et al. (1998) EMBO J. 17:1497-506). The use of these methods suggests that control probes that measure the quality of the processing and the amount of degradation of the sample preferably should be included in the oligonucleotide array. Examples of such control probes are oligonucleotides exactly complementary to 3′- and 5′-ends of constitutively expressed genes, such as beta-actin, transferrin receptor and GAPDH, as mentioned above. The resulting 3′ to 5′ expression ratio of a constitutively expressed gene is then indicative of the quality of processing and the amount of degradation of the sample; i.e., a 3′ to 5′ ratio greater than three (3) indicates either incomplete processing or high RNA degradation (Auer et al. (2003) Nat. Genet. 35:292-93). Consequently, in a preferred embodiment of the invention, the oligonucleotide array includes control probes that are complementary to the 3′- and 5′-ends of constitutively expressed genes.

The quality of the pool of target nucleic acids is not only reflected in the processing and degradation of the target nucleic acids, but also in the origin of the target nucleic acids. Contaminating sequences, such as genomic DNA, may interfere with well-known quantification protocols. Consequently, in a preferred embodiment of the invention, the array further comprises oligonucleotide probes exactly complementary to bacterial genes, ribosomal RNAs, and/or genomic intergenic regions to provide a means to control for the quality of the sample preparation. These probes control for the possibility that the pool of target nucleic acids is contaminated with bacterial DNA, non-mRNA species, and genomic DNA. Exemplary control sequences are set forth as SEQ ID NOs:3576-3642, and are listed in Table 2. As noted above, exemplary tiling sequences for these control sequences are set forth as SEQ ID NOs:7218-7284, and are listed in Table 5.

In a preferred embodiment of the invention, the oligonucleotide array further comprises control mismatch oligonucleotide probes for each perfect match probe. The mismatch probes control for hybridization specificity. Preferably, mismatch control probes are identical to their corresponding perfect match probes with the exception of one or more substituted bases. More preferably, the substitution(s) occurs at a central location on the probe. For example, where a perfect match probe is 25 oligonucleotides in length, a corresponding mismatch probe will have the identical length and sequence except for a single-base substitution at position 13 (e.g., substitution of a thymine for an adenine, an adenine for a thymine, a cytosine for a guanine, or a guanine for a cytosine). The presence of one or more mismatch bases in the mismatch oligonucleotide probe disallows target nucleic acids that bind to complementary perfect match probes to bind to corresponding mismatch control probes under appropriate conditions. Therefore, mismatch oligonucleotide probes indicate whether the incubation conditions are optimal, i.e., whether the stringency being utilized provides for target nucleic acids binding to only exactly complementary probes present in the array.

For each template, a set of perfect match probes exactly complementary to subsequences of consensus, transgene, and/or control sequences (or tiling regions thereof) may be chosen using a variety of strategies. It is known to one of skill in the art that each template can provide for a potentially large number of probes. Also known to one of skill in the art, apparent probes are sometimes not suitable for inclusion in the array. This can be due to the existence of similar subsequences in other regions of the genome, which causes probes directed to these subsequences to cross-hybridize and give false signals. Another reason some apparent probes may not be suitable for inclusion in the array is because they may form secondary structures that prevent efficient hybridization. Finally, hybridization of target nucleic acids with (or to) an array comprising a large number of probes requires that each of the probes hybridizes to its specific target nucleic acid sequence under the same incubation conditions.

An oligonucleotide array may comprise one perfect match probe for a consensus, transgene, or control sequence, or may comprise a probeset (i.e., more than one perfect match probe) for a consensus, transgene, or control sequence. For example, an oligonucleotide array may comprise 1, 5, 10, 25, 50, 100, or more than 100 different perfect match probes for a consensus, transgene or control sequence. In a preferred embodiment of the invention, the array comprises at least 11-150 different perfect match oligonucleotide probes exactly complementary to subsequences of each consensus and transgene sequence. In an even more preferred embodiment, only the most optimal probeset for each template is included. The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the GCG. Any method or software program known in the art may be used to prepare probes for the template sequences of the present invention. For example, oligonucleotide probes may be generated by using Array Designer, a software package provided by TeleChem International, Inc (Sunnyvale, Calif. 94089). Another exemplary algorithm for choosing optimal probesets is described in U.S. Pat. No. 6,040,138.

As disclosed in U.S. Pat. No. 6,040,138, probeset optimization can involve two rounds of selection. In the first round, only perfect match probes that have high stringency requirements (e.g., perfect match probes that will hybridize only with target nucleic acids that are exactly complementary) are selected. These perfect match probes are selected by hybridizing the oligonucleotide array to a sample containing target nucleic acids having subsequences complementary to the oligonucleotide probes, determining the hybridization intensity between each perfect match probe and its corresponding mismatch probe, and selecting perfect match probes that demonstrate a threshold difference in hybridization intensity compared to their corresponding mismatch probe. One of skill in the art will appreciate that this round of selection will ensure that a target nucleic acid sequence will bind only to a complementary perfect match probe and not the corresponding mismatch probe.

In the second round, perfect match oligonucleotide probes and corresponding mismatch probes that demonstrate minimal nonspecific binding are selected. Perfect match probes and corresponding mismatch probes are selected for their specificity by hybridizing the oligonucleotide array with a pool of target nucleic acids that does not contain sequences complementary to the probes, and selecting only those probes in which both the probe and its mismatch control show hybridization intensities below a threshold value. One of skill in the art will appreciate that this second round of selection will ensure that each perfect match probe selected (and corresponding mismatch probe) is unique within the array. Thus, for example, even if the transgene sequences were not included in the initial clustering and alignment analysis, the second round of selection will ensure that oligonucleotide probes to the transgene sequences are complementary only to the transgene sequences.

One of skill in the art will recognize that although the algorithm for oligonucleotide probe selection described in U.S. Pat. No. 6,040,138 will yield a model array of oligonucleotides, it may prove to be extremely costly and time-consuming, especially when a set of perfect match probes must be chosen for a large number of consensus, transgene, and/or control sequences, or tiling regions thereof. Other suitable means to optimize probesets, which will result in a comparable oligonucleotide array, are well known in the art and may be found in, e.g., Lockhart et al. (1996) Nat. Biotechnol. 14:1675-80 and Mei et al. (2003) Proc. Natl. Acad. Sci. USA 100:11237-42.

The oligonucleotide probes of the present invention can be synthesized using a variety of methods. Examples of these methods include, but are not limited to, the use of automated or high throughput DNA synthesizers, such as those provided by Millipore, GeneMachines, and BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or any combination thereof.

Oligonucleotide probes of the present invention may be used in methods of 1) verifying expression of genes, including previously undiscovered genes and/or transgenes, by a cell or cell line and/or 2) determining genes and related pathways involved with conferring a particular cell phenotype, e.g., increased transgene expression, in a sample of interest. Suitable methods for this purpose include, but are not limited to, oligonucleotide arrays (including bead arrays), Southern blot, Northern blot, PCR, and RT-PCR. A sample of interest can be, without limitation, a food sample, an environmental sample, a pharmaceutical sample, a bacterial culture, a clinical sample, a chemical sample, or a biological sample. Examples of biological samples include, but are not limited to, any body fluid, including blood or any of its components (plasma, serum, etc.), menses, mucous, sweat, tears, urine, feces, saliva, sputum, semen, urogenital secretions, gastric washes, pericardial or peritoneal fluids or washes, a throat swab, pleural washes, ear wax, hair, skin cells, nails, mucous membranes, amniotic fluid, vaginal secretions or any other secretions from the body, spinal fluid, human breath, gas samples containing body odors, flatulence or other gases, any biological tissue or matter, or an extractive or suspension of any of these.

III) Forming an Oligonucleotide Array

The methods described above enable an investigator to identify consensus sequences for undiscovered genes in a cell derived from an unsequenced organism, and select probes for that consensus sequence. Thus it is part of the invention that oligonucleotide probes of the present invention can be used to make oligonucleotide arrays that may be used to 1) verify expression of sequences or subsequences of previously undiscovered genes expressed by the cell line and/or 2) determine the involvement in conferring a particular cell phenotype of previously undiscovered genes and/or previously known genes that were not expected to be involved in conferring the particular cell phenotype.

Generally, an array of the invention directed toward an unsequenced organism comprises a first plurality of oligonucleotide probes, each of which is specific to one of a plurality of template sequences, wherein the plurality of template sequences comprises at least one consensus sequence for a gene expressed by a cell derived from the unsequenced organism. As described above, the at least one consensus sequence may be derived from nucleic acid sequences obtained from two different genera and/or species of the organism. In a preferred embodiment, the unsequenced organism is a hamster. In another embodiment, the at least one consensus sequence is selected from the group consisting of the polynucleotide sequences of SEQ ID NOs:19-3572, SEQ ID NOs:3661-7214, complements thereof, and subsequences thereof.

In still another embodiment, an oligonucleotide array of the present invention includes at least 2, 3, 4, 5, 10, 20, 50, 100, 200 or more different probes or probesets, each of which is capable of hybridizing to a template sequence selected from the same Table, e.g., a table in this disclosure, e.g., Table 2. These probes or probesets can be positioned in the same or different discrete regions on the oligonucleotide array. As used herein, two polynucleotides, probes, probesets, etc. are “different” if they have different nucleic acid sequences.

In yet another embodiment, an oligonucleotide array of the present invention includes polynucleotide includes at least 1, 2, 5, 10, 20, 30, 40, 50, 100, 200, 500, 1,000, 2,000, 3,000, or more different probes or probesets, each of which can hybridize under stringent or oligonucleotide array hybridization conditions to a different respective consensus sequence selected from the group consisting of the polynucleotide sequences of SEQ ID NOs:19-3572, SEQ ID NOs:3661-7214, complements thereof, and subsequences thereof.

The length of each probe employed in the present invention can be selected to achieve the desired hybridization effect. For instance, a probe can include or consist of about 15, 20, 25, 30, 35, 40, 45, 50, 60, 70, 80, 90, 100, 200, 300, 400 or more consecutive nucleotides.

Multiple probes for the same template sequence can be included in an oligonucleotide array of the present invention. For instance, at least 2, 5, 10, 15, 20, 25, 30 or more different probes can be used for detecting the same sequence. Each of these different probes can be attached to a different respective region on the oligonucleotide array. Alternatively, two or more different probes can be attached to the same discrete region. The concentration of one probe with respect to the other probe or probes in the same discrete region may vary according to the objectives and requirements of the particular experiment. In one embodiment, different probes in the same region are present in approximately equimolar ratio.

The oligonucleotide arrays of the present invention can also include control probes that can hybridize under stringent or oligonucleotide array hybridization conditions to respective control sequences, or the complements thereof.

The oligonucleotide arrays of the present invention can further include mismatch probes as controls. In many instances, the mismatch residue in each mismatch probe is located near the center of the probe such that the mismatch is more likely to destabilize the duplex with the target sequence under the hybridization conditions. In one embodiment, each mismatch probe on an oligonucleotide array of the present invention is a perfect mismatch probe, and is stably attached to a discrete region different from that of the corresponding perfect match probe.

In many embodiments, the oligonucleotide arrays of the present invention include at least one substrate support that has a plurality of discrete regions. The location of each of these discrete regions is either known or determinable. The discrete regions can be organized in various forms or patterns. For instance, the discrete regions can be arranged as an array of regularly spaced areas on a surface of the substrate. Other regular or irregular patterns, such as linear, concentric or spiral patterns, may also be used.

Oligonucleotide probes may be stably attached to respective discrete regions through covalent or noncovalent interactions. As used herein, an oligonucleotide probe is “stably” attached to a discrete region if the oligonucleotide probe retains its position relative to the discrete region during oligonucleotide array hybridization.

The oligonucleotide array may be immobilized on a solid-phase support, where each oligonucleotide probe is immobilized to a predefined location on the solid-phase support with methods well known in the art such as, but not limited to, very large-scale immobilized polymer synthesis (VLSIP™) technology. VLSIP™ technology immobilizes each oligonucleotide probe in an array of oligonucleotide probes to a predefined location on a solid-phase support using methods including, but not limited to, light-directed coupling, mechanically directed flow paths, spotting on predefined regions, or any combination thereof. These methods are disclosed in U.S. Pat. Nos. 5,143,854; 5,677,195; 5,384,261; 6,040,138; and Fodor et al. (1991) Science 251:767-77, all of which are incorporated herein in their entirety by reference. Any method may be used to attach oligonucleotide probes to an oligonucleotide array of the present invention. In one embodiment, oligonucleotide probes are covalently attached to a substrate support by first depositing the oligonucleotide probes to respective discrete regions on a surface of the substrate support and then exposing the surface to a solution of a cross-linking agent, such as glutaraldehyde, borohydride, or other bifunctional agents. In another embodiment, oligonucleotide probes are covalently bound to a substrate via an alkylamino-linker group or by coating a substrate (e.g., a glass slide) with polyethylenimine followed by activation with cyanuric chloride for coupling the polynucleotides. In yet another embodiment, oligonucleotide probes are covalently attached to an oligonucleotide array through polymer linkers. The polymer linkers may improve the accessibility of the probes to their purported targets. In many cases, the polymer linkers do not significantly interfere with the interactions between the probes and their purported targets.

Oligonucleotide probes may also be stably attached to an oligonucleotide array through noncovalent interactions. In one embodiment, oligonucleotide probes are attached to a substrate support through electrostatic interactions between positively charged surface groups and the negatively charged probes. In another embodiment, a substrate employed in the present invention is a glass slide having a coating of a polycationic polymer on its surface, such as a cationic polypeptide. The oligonucleotide probes are bound to these polycationic polymers. In yet another embodiment, the methods described in U.S. Pat. No. 6,440,723, which is incorporated herein by reference, are used to stably attach oligonucleotide probes to an oligonucleotide array of the present invention.

Numerous materials may be used to make the substrate support(s) of an oligonucleotide array. Suitable materials include, but are not limited to, glass, silica, ceramics, nylon, quartz wafers, gels, metals, and paper. The substrate supports can be flexible or rigid. In one embodiment, they are in the form of a tape that is wound up on a reel or cassette. An oligonucleotide array can include two or more substrate supports. In many embodiments, the substrate supports are nonreactive with reagents that are used in oligonucleotide array hybridization.

The surface(s) of a substrate support may be smooth and substantially planar. The surface(s) of a substrate support can also have a variety of configurations, such as raised or depressed regions, trenches, v-grooves, mesa structures, or other regular or irregular configurations. The surface(s) of the substrate may be coated with one or more modification layers. Suitable modification layers include inorganic or organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on an oligonucleotide array of the present invention may be of any size, shape and density. For instance, they can be squares, ellipsoids, rectangles, triangles, circles, or other regular or irregular geometric shapes, or any portion or combination thereof. In one embodiment, each of the discrete regions has a surface area of less than 10⁻¹ cm², such as less than 10⁻², 10⁻³, 10⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions may range, for example, between 50 and 50,000 regions/cm².

A variety of methods may be used to make the oligonucleotide arrays of the present invention. For instance, the probes can be synthesized in a step-by-step manner on a substrate, or can be attached to a substrate in presynthesized forms. Algorithms for reducing the number of synthesis cycles can be used. In one embodiment, an oligonucleotide array of the present invention is synthesized in a combinational fashion by delivering monomers to the discrete regions through mechanically constrained flowpaths. In another embodiment, an oligonucleotide array of the present invention is synthesized by spotting monomer reagents onto a substrate support using an ink jet printer (such as the DeskWriter C manufactured by Hewlett-Packard). In yet another embodiment, oligonucleotide probes are immobilized on an oligonucleotide array by using photolithography techniques.

Bead arrays and any other type of biochips are also contemplated by the present invention. A bead array comprises a plurality of beads, with each bead stably associated with one or more oligonucleotide probes of the present invention.

Probes for different genes are typically attached to different respective regions on an oligonucleotide array. In certain applications, probes for different genes are attached to the same discrete region.

Methods of Using an Array of the Invention

The nucleic acids arrays of the present invention may be used to 1) verify expression of genes, including previously undiscovered genes and/or transgenes, by a cell or cell line and/or 2) determine genes and related pathways involved with conferring a particular cell phenotype, e.g., increased transgene expression, in a sample of interest. Numerous protocols are available for performing oligonucleotide array analysis. Exemplary protocols include, but are not limited to, those described in GENECHIP® EXPRESSION ANALYSIS TECHNICAL MANUAL (701021 rev. 3, Affymetrix, Inc. 2002). Briefly, such methods comprises the steps of preparing target nucleic acids (which may be RNA or DNA (e.g., genomic DNA, cDNA, etc.)) from a sample of interest, forming a hybridization profile by incubating target nucleic acids with an array, and detecting the hybridization profile (which may or may not include evaluating the hybridization profile). Each of these steps is discussed below. A skilled artisan will recognize that target nucleic acids may not need to be prepared before being used to form a hybridization profile, e.g., already prepared target nucleic acids may be received by an investigator.

I) Preparation of Pool of Target Nucleic Acids

One of skill in the art will recognize that because the above-identified consensus and transgene sequences are derived from known and predicted gene coding sequences, the pool of target nucleic acids (i.e., mRNA or nucleic acids derived therefrom) should reflect the transcription of these regions. Consequently, any biological sample may be used as a source of target nucleic acids. The pool of target nucleic acids can be total RNA, or any nucleic acid derived therefrom, including each of the single strands of cDNA made by reverse transcription of the MRNA, or RNA transcribed from the double-stranded cDNA intermediate. Methods of isolating target nucleic acids for analysis with an oligonucleotide array, such as phenol-chloroform extraction, ethanol precipitation, magnetic bead separation, or silica-gel affinity purification, are well known to one of skill in the art.

For example, various methods are available for isolating or enriching RNA. These methods include, but are not limited to, RNeasy kits (provided by Qiagen), MasterPure kits (provided by Epicentre Technologies), charge-switch technology (see, e.g., U.S. Published patent application Nos. 2003/0054395 and 2003/0130499), and TRIZOL (provided by Gibco BRL). The RNA isolation protocols provided by Affymetrix can also be employed in the present invention. See, e.g., GENECHIP® EXPRESSION ANALYSIS TECHNICAL MANUAL (701021 rev. 3, Affymetrix, Inc. 2002).

In one example, MRNA is enriched by removing rRNA. Different methods are available for eliminating or reducing the amount of rRNA in a sample. For instance, rRNA can be removed by enzyme digestions. According to the latter method, rRNAs are first amplified using reverse transcriptase and specific primers to produce cDNA. The rRNA is allowed to anneal with the cDNA. The sample is then treated with RNAase H, which specifically digests RNA within an RNA:DNA hybrid.

Target nucleic acids may be amplified before incubation with an oligonucleotide array. Suitable amplification methods, including, but not limited to, reverse transcription-polymerase chain reaction, ligase chain reaction, self-sustained sequence replication, and in vitro transcription, are well known in the art. It should be noted that oligonucleotide probes are chosen to be complementary to target nucleic acids. Therefore, if an antisense pool of target nucleic acids is provided (as is often the case when target nucleic acids are amplified by in vitro transcription), the oligonucleotide probes should correspond with subsequences of the sense complement. Conversely, if the pool of target nucleic acids is sense, the oligonucleotide array should be complementary (i.e., antisense) to them. Finally, if target nucleic acids are double-stranded, oligonucleotide probes can be sense or antisense.

The present invention involves detecting the hybridization intensity between target nucleic acids and complementary oligonucleotide probes. To accomplish this, target nucleic acids may be attached directly or indirectly with appropriate and detectable labels. Direct labels are detectable labels that are directly attached to or incorporated into target nucleic acids. Indirect labels are attached to polynucleotides after hybridization, often by attaching to a binding moiety that was attached to the target nucleic acids prior to hybridization. Such direct and indirect labels are well known in the art. In a preferred embodiment of the invention, target nucleic acids are detected using the biotin-streptavidin-PE coupling system, where biotin is incorporated into target nucleic acids and hybridization is detected by the binding of streptavidin-PE to biotin.

Target nucleic acids may be labeled before, during or after incubation with an oligonucleotide array. Preferably, the target nucleic acids are labeled before incubation. Labels may be incorporated during the amplification step by using nucleotides that are already labeled (e.g., biotin-coupled dUTP or dCTP) in the reaction. Alternatively, a label may be added directly to the original nucleic acid sample (e.g., mRNA, cDNA) or to the amplification product after the amplification is completed. Means of attaching labels to nucleic acids are well known to those of skill in the art and include, but are not limited to, nick translation, end-labeling, and ligation of target nucleic acids to a nucleic acid linker to join it to a label. Alternatively, several kits specifically designed for isolating and preparing target nucleic acids for microarray analysis are commercially available, including, but not limited to, the GeneChip® IVT Labeling Kit (Affymetrix, Santa Clara, Calif.) and the Bioarray™ High Yield™ RNA Transcript Labeling Kit with Fluorescein-UTP for Nucleic Acid Arrays (Enzo Life Sciences, Inc., Farmingdale, N.Y.).

Polynucleotides can be fragmented before being labeled with detectable moieties. Exemplary methods for fragmentation include, but are not limited to, heat or ion-mediated hydrolysis.

II) Incubation of Target Nucleic Acids with an Array to Form a Hybridization Profile

Incubation reactions can be performed in absolute or differential hybridization formats. In the absolute hybridization format, polynucleotides derived from one sample are hybridized to the probes in an oligonucleotide array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to an oligonucleotide array. The oligonucleotide array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

In the present invention, the incubation conditions should be such that target nucleic acids hybridize only to oligonucleotide probes that have a high degree of complementarity. In a preferred embodiment, this is accomplished by incubating the pool of target nucleic acids with an oligonucleotide array under a low stringency condition to ensure hybridization, and then performing washes at successively higher stringencies until the desired level of hybridization specificity is reached. In other embodiments, target nucleic acids are incubated with an array of the invention under stringent or well-known oligonucleotide array hybridization conditions. In many examples, these oligonucleotide array hybridization conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6×SSPET, which contains 0.9 M NaCl, 60 mM NaH₂PO₄, 6 mM EDTA, and 0.005% Triton X-100. Under more stringent oligonucleotide array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

III) Detecting Methods

Methods used to detect the hybridization profile of target nucleic acids with oligonucleotide probes are well known in the art. In particular, means of detecting and recording fluorescence of each individual target nucleic acid-oligonucleotide probe hybrid have been well established and are well known in the art, described in, e.g., U.S. Pat. No. 5,631,734, incorporated herein in its entirety by reference. For example, a confocal microscope can be controlled by a computer to automatically detect the hybridization profile of the entire array. Additionally, as a further nonlimiting example, the microscope can be equipped with a phototransducer attached to a data acquisition system to automatically record the fluorescence signal produced by each individual hybrid.

It will be appreciated by one of skill in the art that evaluation of the hybridization profile is dependent on the composition of the array, i.e., which oligonucleotide probes were included for analysis. For example, where the array includes oligonucleotide probes to consensus sequences only, or consensus sequences and transgene sequences only, (i.e., the array does not include control probes to normalize for variation between experiments, samples, stringency requirements, and preparations of target nucleic acids), the hybridization profile is evaluated by measuring the absolute signal intensity of each location on the array. Alternatively, the mean, trimmed mean (i.e., the mean signal intensity of all probes after 2-5% of the probesets with the lowest and highest signal intensities are removed), or median signal intensity of the array may be scaled to a preset target value to generate a scaling factor, which will subsequently be applied to each probeset on the array to generate a normalized expression value for each gene (see, e.g., Affymetrix (2000) Expression Analysis Technical Manual, pp. A5-14). Conversely, where the array further comprises control oligonucleotide probes, the resulting hybridization profile is evaluated by normalizing the absolute signal intensity of each location occupied by a test oligonucleotide probe by means of mathematical manipulations with the absolute signal intensity of each location occupied by a control oligonucleotide probe. Typical normalization strategies are well known in the art, and are included, for example, in U.S. Pat. No. 6,040,138 and Hill et al. (2001) Genome Biol. 2(12):research0055.1-0055.13.

Signals gathered from oligonucleotide arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subjected to further analysis. For instance, the hybridization signal for each probe can be normalized to take into account variations in hybridization intensities when more than one array is used under similar test conditions. Signals for individual target nucleic acids hybridized with complementary probes can also be normalized using the intensities derived from internal normalization controls contained on each array. In addition, genes with relatively consistent expression levels across the samples can be used to normalize the expression levels of other genes.

Applications

The invention also involves using the above-described oligonucleotide array and related methods to optimize culture conditions for a particular cell line, identify genes (including previously undiscovered genes) and/or gene pathways that confer a particular cell-line phenotype, and determine overall cellular productivity for either intrinsic proteins or extrinsic proteins (e.g., those encoded by transgenes). The oligonucleotide array described above can be used to optimize culture conditions by first establishing a database of hybridization profiles, each of which correlates to a different set of culture conditions. For example, a first sample obtained from cells grown in normal culture conditions can be analyzed using the oligonucleotide array and methods described herein. The resulting hybridization profile will reflect the baseline expression of genes when the particular cell line is grown in normal conditions. A second sample obtained from cells grown under conditions that induce, e.g., a stress response, such as cells grown at a high temperature, can be analyzed using the oligonucleotide array and methods described herein. The resulting hybridization profile from the second sample likely will be different than that obtained from the first sample. A third sample obtained from cells cultured in yet another condition that induces a stress response, such as cells grown in the absence of serum, will result in yet another hybridization profile distinct from those obtained from the first and second samples. The process of obtaining the hybridization profiles of samples from cells grown in different culture conditions can be continued such that a particular hybridization profile will reflect that the cells were grown in a particular culture condition. With such a database, one of skill in the art can readily determine in what culture conditions (e.g., stress-inducing conditions) the cells used in an experiment were grown. Other factors, in addition to temperature, that contribute to stress-inducing culture conditions include, but are not limited to, serum concentration, nutrient concentration, metabolite concentration, pH, lactate concentration, ammonia concentration, oxidation level, sodium butyrate concentration, valeric acid concentration, hexamethylene bisacetamide concentration, cell concentration, cell viability, and recombinant protein concentration in actively growing or stationary cultures.

In establishing this database, the different genes and genetic pathways that are regulated during different conditions will be elucidated. The array described herein will be particularly useful in identifying previously undiscovered genes or genetic pathways. For example, whereas it is established that changes in the temperature of the culture will generally result in the overexpression of certain known genes (e.g., an increased temperature results in overexpression of certain heat-shock proteins), it is likely that temperature-related stresses will induce/reduce the expression of other genes, including previously undiscovered genes and even perhaps previously known genes not obviously related to stress responses. Analysis of a cell line grown in varying temperatures using the array of oligonucleotides and related methods of this invention will identify these previously known and unknown genes because the oligonucleotide array is designed to include known and previously undiscovered gene coding sequences.

Similarly, the above methods can be used to identify genes that confer or correlate with a desired phenotype or characteristic. As nonlimiting examples, desired phenotypes or characteristics may be conferred to cells by growing the cells in different temperatures, to a high cell density, to produce a high titer of transgene products with the use of agents such as sodium butyrate, to be in different kinetic phases of growth (e.g., lag phase, exponential growth phase, stationary phase or death phase), and/or to become serum-independent, etc. During the period in which these phenotypes are induced, and/or after these phenotypes are achieved, a pool of target nucleic acid samples can be prepared from the cells and analyzed with the oligonucleotide array to determine and identify which genes demonstrate altered expression in response to a particular stimulus (e.g., temperature, sodium butyrate), and therefore are potentially involved in conferring the desired phenotype or characteristic.

One of skill in the art will appreciate that the methods and associated oligonucleotide arrays described above can be used not only to measure the success or failure of modifying cell lines with a transgene, but also to increase expression of the transgene. For example, to determine whether a cell line has been successfully engineered to express a transgene, target nucleic acids can be prepared from nontransfected and transfected cells. The target nucleic acids can then be hybridized to (e.g., incubated with) an oligonucleotide array that includes probes to transgene sequences. If the resulting hybridization profile demonstrates high signal intensities at the locations of the probes to transgene sequences, the cells have been successfully engineered. If the signal intensities at these locations are low, the cells were either unsuccessfully engineered, or they were successfully engineered but were grown in culture conditions unfavorable to transgene expression. By comparing the resulting hybridization profile with established hybridization profiles that reflect the nature of the culture conditions, it can be determined whether the cells were successfully engineered but grown in suboptimal culture conditions, and the culture conditions can be subsequently changed to increase the expression of the transgene. In another embodiment of the invention, target nucleic acid samples are prepared from transfected cells expressing different levels of the transgene, or grown in different conditions that increase gene expression, and analyzed with the oligonucleotide array to identify specific genes and related genetic pathways that correlate to or confer high transgene expression. The identified genes and related pathways can then be manipulated to induce cell lines to express higher levels of the transgene.

The oligonucleotide arrays of the present invention may also be used to identify or evaluate agents capable of conferring a particular cell phenotype. Any compound-screening method may be used in the present invention. These methods typically include the steps of (1) contacting a molecule of interest with a culture comprising the cell of interest, or administrating the molecule of interest to an animal comprising the cell of interest; and (2) hybridizing nucleic acid molecules prepared from the culture or animal model to an oligonucleotide array of the present invention. Changes in the hybridization signals in the presence of the molecule of interest compared to that in the absence of the molecule can be used to determine the effect of the molecule on the cell of interest. Any type of agent can be evaluated according to the present invention, such as, but not limited to, small molecules, antibodies, peptides, or peptide mimetics.

The methods disclosed herein of making and using oligonucleotide arrays in the optimization of cell line culture conditions and transgene expression may be used for cells from a variety of organisms, including, but not limited to, bacteria, plants, fungi, and animals (the latter including, but not limited to, insects and mammals). As such, embodiments of the invention include methods of making oligonucleotide arrays comprising identifying consensus sequences for known and previously undiscovered genes of, for example, Escherichia coli, Spodopterafrugiperda, Nicotiana sp., Zea maize, Lemna sp., Saccharomyces sp., Pichia sp., Schizosaccharomyces sp., Chinese Hamster Ovary (CHO) cells, and baby hamster kidney (BHK) cells. Other embodiments of the invention include oligonucleotide arrays comprising oligonucleotide probes complementary to consensus sequences for known and previously undiscovered genes of, for example, Escherichia coli, Spodopterafrugiperda, Nicotiana sp., Zea maize, Lemna sp., Saccharomyces sp., Pichia sp., Schizosaccharomyces sp., CHO cells, and BHK cells. Embodiments of the invention also include methods of using oligonucleotide arrays complementary to consensus sequences for known and previously undiscovered genes of, for example, Escherichia coli, Spodoptera frugiperda, Nicotiana sp., Zea maize, Lemna sp., Saccharomyces sp., Pichia sp., Schizosaccharomyces sp., CHO cells, and BHK cells. The above list of organisms and cell lines are meant only to provide nonlimiting examples. As such, oligonucleotide arrays comprising oligonucleotide probes to consensus sequences for known and previously undiscovered genes of any organism, and methods of making and using these arrays, are within the scope of the invention.

Isolated Polynucleotides

In one embodiment of the invention, the inventors aligned gene coding sequences and EST sequences obtained from hamsters, e.g., Cricetulus griseus, Cricetulus migratorius, Mesocricetus auratus, etc., and hamster cell lines, e.g., the CHO cell line, to identify consensus sequences for known and previously undiscovered genes of the CHO cell line (see Example 1.2 and Table 2). Also, the inventors generated perfect match and mismatch probesets for each consensus sequence and, in addition to control probesets, generated an array of all oligonucleotide probes (See Example 1.3). Use of the oligonucleotide array then verified expression of a subset of previously undiscovered gene sequences by CHO cells and identified a second subset of gene sequences that may be used as novel targets to confer a particular cell phenotype, both of which are subsets of the consensus sequences (Table 2). Additionally, use of the oligonucleotide array confirmed the expression of another hamster gene, caspase 8, which was previously undiscovered. Accordingly, the present invention provides polynucleotide sequences (or subsequences) of genes that are newly discovered to be expressed by CHO cells. The invention also provides sequences (or subsequences) of genes that may be used as targets to effect a cell phenotype, particularly a phenotype characterized by increased and efficient production of a recombinant transgene.

Accordingly, the present invention provides novel isolated and purified polynucleotides that are either or both 1) previously undiscovered gene sequences verifiably expressed by CHO cells and 2) sequences involved in regulating a cell phenotype, e.g., transgene expression (and thus may be used as novel targets to increase transgene productivity). It is part of the invention to provide inhibitory polynucleotides to the novel isolated and purified polynucleotides of the invention, particularly to polynucleotides involved in regulating a cell phenotype (e.g., may be used as targets to increase transgene productivity); such inhibitory polynucleotides may be used as antagonists to such previously undiscovered genes.

Thus, the invention provides each purified and isolated polynucleotide sequence selected from Table 2 that is, or is part of, a previously undiscovered gene (i.e., a gene that had not been sequenced and/or shown to be expressed by CHO cells) and is verifiably expressed by CHO cells, herein designated a “novel CHO sequence.” Exemplary, but nonlimiting, novel CHO sequences are listed in Table 3. Preferred DNA sequences of the invention include genomic and cDNA sequences and chemically synthesized DNA sequences. The polynucleotide sequences of cDNAs encoding novel CHO sequences may have and/or consist essentially of a sequence selected from the gene sequences listed in Table 3 and set forth as SEQ ID NOs:3439-3573, and the gene sequences set forth as SEQ ID NOs:7081-7215, SEQ ID NO:3574, and SEQ ID NO:7216.

The invention also provides each purified and isolated polynucleotide sequence selected from Table 2 that is shown to be a suitable target for regulating a CHO cell phenotype, i.e., is differentially expressed by a first population of CHO cells cultured under a first set of conditions compared to a second population of CHO cells cultured under a second set of conditions, herein designated as “differential CHO sequences.” Differential CHO sequences are preferably suitable targets for regulating cell survival under stressful culture conditions, transgene expression by transgene-modified CHO cells, and/or production of potential antigens, e.g., N-glycolylneuraminic acid (NGNA). For example, in a nonlimiting preferred embodiment, a differential CHO sequence may have and/or consist essentially of a sequence selected from the gene sequences listed in Table 4 and set forth as SEQ ID NOs:3421-3572 and the gene sequences set forth as SEQ ID NOs:7063-7214. A skilled artisan will recognize that the differential CHO sequences of the invention may include novel CHO sequences, known gene sequences that are attributed with a function that is, or was, not obviously involved in transgene expression, and known sequences that previously had no known function but may now be known to function as targets in regulating a CHO cell phenotype.

Polynucleotides of the present invention also include polynucleotides that hybridize under stringent conditions to novel and/or differential CHO sequences, or complements thereof, and/or encode polypeptides that retain substantial biological activity of polypeptides encoded by novel and/or differential CHO sequences of the invention. Polynucleotides of the present invention also include continuous portions of novel and/or differential CHO sequences comprising at least 21 consecutive nucleotides.

Polynucleotides of the present invention also include polynucleotides that encode any of the amino acid sequences encoded by the polynucleotides as described above, or continuous portions thereof, and that differ from the polynucleotides described above only due to the well-known degeneracy of the genetic code.

The isolated polynucleotides of the present invention may be used as hybridization probes (e.g., as an oligonucleotide array, as described above) and primers to identify and isolate nucleic acids having sequences identical to, or similar to, those encoding the disclosed polynucleotides. Hybridization methods for identifying and isolating nucleic acids include polymerase chain reaction (PCR), Southern hybridization, and Northern hybridization, and are well known to those skilled in the art.

Hybridization reactions can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table A below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE A

| | Poly- | | Hybridization | |
|---|---|---|---|---|
| Stringency | nucleotide | Hybrid | Temperature and | Wash Temperature |
| Condition | Hybrid | Length (bp)¹ | Buffer² | and Buffer² |
| A | DNA:DNA | >50 | 65° C.; 1 × SSC -or- | 65° C.; 0.3 × SSC |
| | | | 42° C.; 1 × SSC, 50% | |
| | | | formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; 0.3 × SSC |
| | | | 45° C.; 1 × SSC, 50% | |
| | | | formamide | |
| D | DNA:RNA | <50 | T_{D}*; 1 × SSC | T_{D}*; 1 × SSC |
| E | RNA:RNA | >50 | 70° C.; 1 × SSC | 70° C.; O.3 ×SSC |
| | | | -or- | |
| | | | 50° C.; 1 × SSC, 50% | |
| | | | formamide | |
| F | RNA:RNA | <50 | T_{F}*; 1 × SSC | T_{f}*; 1 × SSC |
| G | DNA:DNA | >50 | 65° C.; 4 × SSC | 65° C.; 1 × SSC |
| | | | -or- | |
| | | | 42° C.; 4 × SSC, 50% | |
| | | | formamide | |
| H | DNA:DNA | <50 | T_{H}*; 4 × SSC | T_{H}*; 4 × SSC |
| I | DNA:RNA | >50 | 67° C.; 4 × SSC | 67° C.; 1 × SSC |
| | | | -or- | |
| | | | 45° C.; 4 × SSC, 50% | |
| | | | formamide | |
| J | DNA:RNA | <50 | T_{J}*; 4 × SSC | T_{J}*; 4 × SSC |
| K | RNA:RNA | >50 | 70° C.; 4 × SSC | 67° C.; 1 × SSC |
| | | | -or- | |
| | | | 50° C.; 4 × SSC, 50% | |
| | | | formamide | |
| L | RNA:RNA | <50 | T_{L}*; 2 × SSC | T_{L}*; 2 × SSC |
| M | DNA:DNA | >50 | 50° C.; 4 × SSC | 50° C.; 2 × SSC |
| | | | -or- | |
| | | | 40° C.; 6 × SSC, 50% | |
| | | | formamide | |
| N | DNA:DNA | <50 | T_{N}*; 6 × SSC | T_{N}*; 6 × SSC |
| O | DNA:RNA | >50 | 55° C.; 4 × SSC | 55° C.; 2 × SSC |
| | | | -or- | |
| | | | 42° C.; 6 × SSC, 50% | |
| | | | formamide | |
| P | DNA:RNA | <50 | T_{P}*; 6 × SSC | T_{P}*; 6 × SSC |
| Q | RNA:RNA | >50 | 60° C.; 4 × SSC -or- | 60° C.; 2 × SSC |
| | | | 45° C.; 6 × SSC, 50% | |
| | | | formamide | |
| R | RNA:RNA | <50 | T_{R}*; 4 × SSC | T_{R}*; 4 × SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of | | | | |
| # the polynucleotides and identifying the region or regions of optimal sequence complementarity. | | | | |
| ²SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, | | | | |
| # Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1 × SSC = 0.165 M). | | | | |

Additional examples of stringency conditions for polynucleotide hybridization are provided in Sambrook et al. (1989) Molecular Cloning: A Laboratory Manual, Chs. 9 & 11, Cold Spring Harbor Laboratory Press, Cold Spring Harbor, NY, and Ausubel et al., eds. (1995) Current Protocols in Molecular Biology, Sects. 2.10 & 6.3-6.4, John Wiley & Sons, Inc., herein incorporated by reference.

Generally, and as stated above, the isolated polynucleotides of the present invention may also be used as hybridization probes and primers to identify and isolate DNAs homologous to the disclosed polynucleotides. These homologs are polynucleotides isolated from different species than those of the disclosed polynucleotides, or within the same species, but with significant sequence similarity to the disclosed polynucleotides. Preferably, polynucleotide homologs have at least 60% sequence identity (more preferably, at least 75% identity; most preferably, at least 90% identity) with the disclosed polynucleotides. Preferably, homologs of the disclosed polynucleotides are those isolated from mammalian species.

The isolated polynucleotides of the present invention may also be used as hybridization probes and primers to identify cells and tissues that express the polynucleotides of the present invention and the conditions under which they are expressed.

Additionally, the polynucleotides of the present invention may be used to alter (i.e., regulate (e.g., enhance, reduce, or modify)) the expression of the genes corresponding to the novel and/or differential CHO sequences of the present invention in a cell or organism. These corresponding genes are the genomic DNA sequences of the present invention that are transcribed to produce the mRNAs from which the novel and/or differential CHO polynucleotide sequences of the present invention are derived.

Altered expression of the novel and/or differential CHO sequences encompassed by the present invention in a cell or organism may be achieved through the use of various inhibitory polynucleotides, such as antisense polynucleotides, ribozymes that bind and/or cleave the MRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29).

The inhibitory antisense or ribozyme polynucleotides of the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-70). Alternatively, antisense molecules can be produced biologically using an expression vector into which a polynucleotide of the present invention has been subcloned in an antisense (i.e., reverse) orientation.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) encompassed by the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas MRNA (Song et al. (2003) Nat. Med. 9:347-51). In addition, intrathecally delivered siRNA has recently been reported to block pain responses in two models (agonist-induced pain model and neuropathic pain model) in the rat (Dom et al. (2004) Nucleic Acids Res. 32(5):e49).

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the polynucleotides of the present invention can be designed based on criteria well known in the art (e.g., Elbashir et al. (2001) EMBO J. 20:6877-88). For example, the target segment of the target MRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target mRNA and should be at least 75 bp after the initiation ATG and at least 75 bp before the stop codon. siRNA molecules targeted to the polynucleotides of the present invention can be designed by one of ordinary skill in the art using the aforementioned criteria or other known criteria.

Altered expression of the novel and/or differential CHO genes sequences of the present invention in a cell or organism may also be achieved through the creation of nonhuman transgenic animals into whose genomes polynucleotides of the present invention have been introduced. Such transgenic animals include animals that have multiple copies of a gene (i.e., the transgene) of the present invention. A tissue-specific regulatory sequence(s) may be operably linked to a polynucleotide of present invention to direct its expression to particular cells or a particular developmental stage. In another embodiment, transgenic nonhuman animals can be produced that contain selected systems that allow for regulated expression of the transgene. One example of such a system known in the art is the cre/loxP recombinase system of bacteriophage P1. Methods for generating transgenic animals via embryo manipulation and microinjection, particularly animals such as mice, have become conventional and are well known in the art (e.g., Bockamp et al. (2002) Physiol. Genomics 11:115-32). In preferred embodiments of the invention, the nonhuman transgenic animal comprises at least one novel and/or differential CHO sequence.

Altered expression of the genes of the present invention in a cell or organism may also be achieved through the creation of animals whose endogenous genes corresponding to the polynucleotides of the present invention have been disrupted through insertion of extraneous polynucleotides sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of the present invention may be operably linked to an expression control sequence such as the pMT2 and pED expression vectors for recombinant production of the polypeptides encoded by the polynucleotides of the invention. General methods of expressing recombinant proteins are well known in the art.

A number of cell types may act as suitable host cells for recombinant expression of the polypeptides encoded by the polynucleotides of the invention. Mammalian host cells include, but are not limited to, e.g., COS cells, CHO cells, 293 cells, A431 cells, 3T3 cells, CV-1 cells, HeLa cells, L cells, BHK21 cells, HL-60 cells, U937 cells, HaK cells, Jurkat cells, normal diploid cells, cell strains derived from in vitro culture of primary tissue, and primary explants.

Alternatively, it may be possible to recombinantly produce the polypeptides encoded by polynucleotides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides are made in yeast or bacteria, it may be necessary to modify them by, e.g., phosphorylation or glycosylation of appropriate sites, in order to obtain functionality. Such covalent attachments may be accomplished using well-known chemical or enzymatic methods.

The polypeptides encoded by polynucleotides of the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac^{®}kit, Invitrogen, Carlsbad, Calif.).

Following recombinant expression in the appropriate host cells, the polypeptides encoded by polynucleotides of the present invention may then be purified from culture medium or cell extracts using known purification processes, such as gel filtration and ion exchange chromatography. Purification may also include affinity chromatography with agents known to bind the polypeptides encoded by the polynucleotides of the present invention. These purification processes may also be used to purify the polypeptides from natural sources.

Alternatively, the polypeptides encoded by polynucleotides of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides encoded by polynucleotides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides encoded by polynucleotides of the present invention may also be produced by known conventional chemical synthesis. Methods for chemically synthesizing the polypeptides encoded by polynucleotides of the present invention are well known to those skilled in the art. Such chemically synthetic polypeptides may possess biological properties in common with the natural, purified polypeptides, and thus may be employed as biologically active or immunological substitutes for the natural polypeptides.

Screening Assays and Sources of Test Compounds

The polynucleotides of the present invention, particularly those of differential CHO sequences, may also be used in screening assays to identify pharmacological agents or lead compounds that may be used to regulate the phenotype of CHO cells, e.g., which may be used to increase transgene expression by a transgene-modified CHO cell. For example, different populations of CHO cells can be contacted with one of a plurality of test compounds (e.g., small organic molecules or biological agents), and the expression of at least one differential CHO gene sequence may be compared in untreated samples or in samples contacted with different test compounds to determine whether any of the test compounds provides a substantially modulated (e.g., increased or decreased) level of expression. In a preferred embodiment, the identification of test compounds capable of modulating the activity of at least one differential CHO gene sequence is performed using high-throughput screening assays, such as provided by BIACORE® (Biacore International AB, Uppsala, Sweden), BRET (bioluminescence resonance energy transfer), and FRET (fluorescence resonance energy transfer) assays, as well as ELISA. One of skill in the art will recognize that test compounds capable of decreasing levels of a differential CHO gene sequence(s), particularly a differential CHO gene sequence listed in Table 2, may be an exemplary candidate for increasing transgene expression by CHO cells.

The test compounds of the present invention may be obtained from a number of sources. For example, combinatorial libraries of molecules are available for screening. Using such libraries, thousands of molecules can be performed for inhibitory activity. Preparation and screening of compounds can be performed as described above or by other methods well known to those of skill in the art. The compounds thus identified can serve as conventional “lead compounds” or can be used as the actual therapeutics.

### EXAMPLES

The Examples which follow are set forth to aid in the understanding of the invention but are not intended to, and should not be construed to, limit its scope in any way. The Examples do not include detailed descriptions of conventional methods, such as probe selection, real-time polymerase chain reaction (PCR), photolithography, cell culture, RNA quantification or those methods employed in the construction of vectors, the insertion of genes encoding the polypeptides into such vectors and plasmids, the introduction of such vectors and plasmids into host cells, and the expression of polypeptides from such vectors and plasmids in host cells. Such methods are well known to those of ordinary skill in the art.

### Example 1

### Generation of an Oligonucleotide Array Usefor for Monitoring Gene Expression by Chinese Hamster Ovary Cells

Chinese Hamster Ovary (CHO) cells are commonly used for the recombinant production of proteins. Despite the widespread use of CHO cells in the art, only limited sequence analysis of the cell line has been performed, and methods to monitor CHO cell gene expression are not readily available. Consequently, publicly available gene coding sequences from all hamsters, in addition to gene coding sequences from the Chinese hamster, were clustered and aligned to generate consensus sequences. Chinese hamster gene coding sequences and EST sequences were obtained either from publicly available sources or through use of CHO cDNA libraries made by well-known methods in the art.

### Example 1.1

### Generation of CHO cDNA Library

Generation of a cDNA library is a well-known method in the art. Briefly, a cDNA library is constructed from a source of a pool of MRNA, which is subsequently reverse transcribed into cDNA. The resulting pool of cDNA is then ligated into a population of an appropriate expression vector to form the cDNA library. Well-known methods for efficient cDNA—expression vector ligation, such as tailing, linker/adaptor insertion, and vector priming, are described in the art, e.g., Kriegler, M. P. (1990) Gene Transfer and Expression: A Laboratory Manual, W.H. Freeman and Company, NY, pp. 117-31. Additionally, methods for cDNA library amplification, isolation, and sequencing are also well known in the art.

One of skill in the art will recognize that the source of mRNA depends on the cell line to be monitored, as described above. It is preferred that the mRNA is isolated from either the cells or cell line(s) to be monitored, or the animal from which the cell line was derived. Additionally, if the mRNA is to be isolated from the cell line to be monitored, it is preferable that mRNA be isolated from the cell line grown in various culture conditions to increase the possibility of including EST sequences that are involved in cell growth, cell maintenance, and/or transgene production.

To generate CHO cDNA libraries, mRNA was isolated from cultured CHO cells in both log phase and stationary phase. The libraries containing cDNA inserts within the pBluescriptII vector were normalized to reduce the amount of redundant transcripts (see, e.g., Soares et al. (1994) Proc. Natl. Acad. Sci. USA 91:9228-32; Tanaka et al. (1996) Genomics 35:231-35; Bondaldo et al. (1996) Genome Res. 6:791-806). Aliquots of the libraries were plated to obtain individual cDNA clones. Plasmid DNA from each clone was isolated and sequenced.

### Example 1.2

### Identification of Consensus Sequences

All hamster sequences, either gene coding sequences publicly available from GenBank or generated with prediction algorithms (1,358 sequences) or EST sequences derived from a CHO cDNA library (4,120 sequences) as generated in Example 1.1, were included in a sequence set to be analyzed by clustering and alignment. In a first step, each sequence (i.e., gene coding or EST sequence) of the sequence set was screened for vector and low-complexity sequences. The vector and low-complexity sequences were masked from each gene coding sequence or EST sequence with a poly-X sequence of the same length, and the remaining sequence was either included for clustering and alignment analysis, or excluded because it did not meet the base pair requirement inherent in the preset definition of homologous sequences, e.g., the remaining sequence was 50 base pairs in length whereas the definition of homologous sequences required at least 100 base pairs. The base pair requirement may be preset by one of skill in the art to remove sequences containing, for example, less than 1-150 bases (after screening).

The sequence set was analyzed with the clustering and alignment tool CAT (DoubleTwist, Oakland, Calif.), which first masked low-complexity regions and then reduced the redundancy of the sequence set based on user-defined parameters that required the sequences to be 100 or more base pairs in length. The resulting sequence set derived from CAT contained two distinct groups of consensus sequences. The first group was a set of consensus sequences for CAT subclusters containing more than one sequence. Hypothetically, the multi-sequence subclusters represented single transcripts included in the input sequence set numerous times. The second group was a set of exemplar (i.e., singleton) sequences that did not cluster with other CAT subclusters.

Of an original 5,478 input sequences, 601 sequences were removed as a result of screening. The remaining 4,877 sequences were processed through CAT. Initial clustering was performed at a minimum threshold of ninety percent sequence identity over a 100 base pair region. Sequence alignment was performed with Phrap (University of Washington, Seattle, Wash.) using default alignment criteria. The above cluster and alignment analysis produced 3,553 consensus sequences (601 of the consensus sequences were derived from multi-sequence clusters and 2,952 of the consensus sequences were derived from singleton clusters). The consensus sequences are set forth as SEQ ID NOs:19-3572.

An example of a multi-sequence subcluster and its corresponding consensus sequence is provided in FIG. 1. The sequence of ribosomal protein L13 from Chinese Hamster Ovary cells (available from GenBank; Accession no. AB014876) clustered with two expressed sequence tags obtained from the CHO cDNA library. As shown in FIG. 1, alignment analysis of the three sequences revealed two areas of low complexity and one area of low homology. The two areas of low complexity, as well as an area containing contaminating vector sequence, were masked with a series of X's. The area of low homology is spanned by what is designated in the consensus sequence by a K (position 137) and an R (position 158) (letter designation following traditional IUPAC notation). The resulting consensus sequence was oriented 5′ to 3′ as determined from the original GenBank records of the known genes and/or through the presence of an internal 3′ read generated with the CHO library for the previously undiscovered genes, and used as a template for the selection of oligonucleotide probes.

### Example 1.3

### Probe Selection

Tiling regions of (1) consensus sequences identified in Example 1.2 and set forth as SEQ ID NOs:19-3572, (2) transgene sequences including those listed in Table 1 and set forth as SEQ ID NOs:1-18, (3) other hamster sequences set forth as SEQ ID NOs:3573-3575, and (4) control sequences (set forth as SEQ ID NOs:3576-3642) as described above, were subject to a first stage of probe selection analysis during which every potential 25-mer perfect match oligonucleotide probe was identified for each consensus, transgene and control sequence. The sequences for the tiling regions of the sequences are set forth as SEQ ID NOs:3643-7284, wherein the sequence of SEQ ID NO:3642+n corresponds to the tiling sequence for the sequence set forth in SEQ ID NO:n. In addition, a 25-mer oligonucleotide probe with a single mutation in the 13^{th} position (mismatch) was generated for each perfect match oligonucleotide probe.

The perfect match and mismatch probes were analyzed for, and scored based on, their stringency requirements and inherent structures. In a second stage of probe selection, probe sequences were determined to be either unique or multiply represented with respect to all other probe sequences identified in the first stage. Finally, probesets for each consensus, transgene and control sequence were created such that each probe in a probeset had a similar characteristic with regard to its score (derived in the first stage of probe selection) and uniqueness (determined in the second stage of probe selection). Four distinct classes of probesets of at least 25-55 perfect match 25-mer oligonucleotide probes were designed for each consensus, transgene and control sequence. Following is a description of the four classes of probesets in the order of suitability for inclusion in the array: 1) probesets consisting of high-scoring, unique probes; 2) probesets consisting of lower-scoring, unique probes; 3) probesets consisting of high-scoring, nonunique probes where every probe can be used for detection of a small set of highly homologous sequences; and 4) probesets consisting of high-scoring, unique and nonunique probes where at least one probe is specific for the identified sequence and the remaining probes in the probeset are common to a small set of highly homologous sequences. If a probeset fell within the first class of probesets, i.e., the probes within the probeset were high-scoring and unique, no probeset within the other three classes of probesets were incorporated into the array design. Finally, if none of the four classes of probesets could be designed for a particular sequence, the array would not contain a probeset for that sequence, and thus, the sequence would not be detectable with the array. As demonstrated in FIG. 1, probes were not generated for areas of low homology, low complexity, or areas containing contaminating vector sequences. All oligonucleotide probes were then arrayed onto a solid phase substrate in a random but known location by photolithography.

### Example 2

### Hybridization of a Pool of Target Nucleic Acids to the Oligonucleotide Array and Detection of the Hybridization Profile

The following example is applicable to any sample obtained from any cell line cultured in a particular condition. In other words, the protocols described in this example can be used to obtain a hybridization profile for nontransfected cells, cells transfected with a transgene, and nontransfected or transfected cells grown in differing culture conditions.

### Example 2.1

### Preparing a Pool of Target Nucleic Acids

Using well-known methods in the art, total RNA was isolated from the sample and converted to biotinylated cRNA for hybridization to the oligonucleotide array made in Example 1. Briefly, total RNA was isolated using the RNeasy Kit (Qiagen, Valencia, Calif.) according to the manufacturer's protocol. The isolated total RNA (5 μg) was then annealed to an oligo-dT primer (50 pMoles) in a reaction containing the BAC pool control reagent by incubation at 70° C. for 10 min. The primed RNA was subsequently reverse transcribed into complementary DNA (cDNA) by incubation with 200 units of Superscript RT II™ (Invitrogen, Carlsbad, Calif.) and 0.5 mM each dNTP (Invitrogen) in 1× first-strand buffer at 50° C. for 1 hr. Second-strand synthesis was performed by the addition of 40 units DNA Pol I, 10 units E. coli DNA ligase, 2 units RNase H, 30 μl second-strand buffer (Invitrogen), 3 μl of 10 mM dNTP (2.5 mM each) and dH₂O to a 150 μl final volume and incubation at 15° C. for 2 hours. T4 DNA polymerase (10 units) was then added for an additional 5 min. The reaction was stopped by the addition of 10 μl of 500 mM EDTA. The resulting double-stranded cDNA was purified using a cDNA Sample Cleanup Module (Affymetrix). The cDNA (3 μl) was transcribed in vitro into cRNA by incubation with 1750 units of T7 RNA polymerase and biotinylated rNTPs at 37° C. for 16-20 hrs. Biotinylated rNTPs were used to incorporate biotin into the resulting cRNA. The biotinylated cRNA was then purified using the cRNA Sample Cleanup Module (Affymetrix) according to the manufacturer's protocol, and quantified using a spectrophotometer.

### Example 2.2

### Hybridization of a Pool of Target Nucleic Acids to Oligonucleotide Array

Biotin-labeled cRNA (2.5 μg) was fragmented for 35 min at 95° C. in 40 μl of 1× Fragmentation Buffer (Affymetrix). The fragmented cRNA was diluted in hybridization fluid [260 μl 1× MES buffer containing 300 ng herring sperm DNA, 300 ng BSA, 6.25 μl of a control oligonucleotide used to align the oligonucleotide array (e.g., Oligo B2, commercially available from Affymetrix, used to align Affymetrix arrays of oligonucleotide probes), and 2.5 μl standard curve reagent (as described in Hill et al. (2000) Science 290:809-12)] and denatured for 5 min at 95° C., followed immediately by incubation for 5 min at 45° C. Insoluble material was removed by a brief centrifugation, and the hybridization mix was added to the oligonucleotide array described in Example 1. Target nucleic acids were allowed to hybridize to complementary oligonucleotide probes by incubation at 45° C. for 16 hrs under continuous rotation at 60 rpm. After incubation, the hybridization fluid was removed and the oligonucleotide array was extensively washed with 6×SSPET and 1×SSPET using protocols known in the art.

### Example 2.3

### Detection and Analysis of the Hybridization Profile Resulting from Hybridizing the Pool of Target Nucleic Acids to the Oligonucleotide Array

The raw fluorescent intensity value of each gene was measured at a resolution of 3 μm with an Agilent GeneArray Scanner. Microarray Suite (Affymetrix, Santa Clara, Calif.), which uses an algorithm to determine whether a gene is “present” or “absent,” as well as the specific. hybridization intensity values of each gene on the array, was used to evaluate the fluorescent data. The expression value for each gene was normalized to frequency values by referral to the expression value of 11 control transcripts of known abundance that were spiked into each hybridization mix according to the procedure of Hill et al. (2001) Genome Biol. 2(12):research0055.1-0055.13 and Hill et al. (2000), Science 290:809-12, both of which are incorporated herein in their entirety by reference. The frequency of each gene was calculated and represents a value equal to the total number of individual gene transcripts per 10⁶ total transcripts.

Each condition and time point was represented by at least three biological replicates. Programs known in the art, e.g., GeneExpress 2000 (Gene Logic, Gaithersburg, Md.), were used to analyze the presence or absence of a target sequence and to determine its relative expression level in one cohort of samples (e.g., condition or time point) compared to another sample cohort. A probeset called present in all replicate samples was considered for further analysis. Generally, fold-change values of 2-fold or greater were considered statistically significant if the p-values were less than or equal to 0.05.

### Example 3

### Use of the Oligonucleotide Array to Identify Genes and Related Pathways Involved with a Particular Cell Phenotype

The identification of genes and related pathways that are involved with one or more particular cell phenotypes (e.g., during a stress response, transgene expression, etc.) can lead to the discovery of genes that were previously undiscovered, e.g., as indicators of a stress-inducing culture condition, involvement with expression of a transgene, etc., respectively. One of skill in the art may identify the genes and related pathways involved in particular cell phenotypes by performing the following:
1) creating a plurality of identical oligonucleotide arrays for the cells (as described in Example 1);
2) growing a first sample of cells in a first condition that mimics the physiological condition and growing a second sample of cells in a second condition that induces a particular cell phenotype;
3) isolating, processing, and hybridizing total RNA from the first sample to a first oligonucleotide array created in step 1 (as described in Example 2);
4) isolating, processing, and hybridizing total RNA from the second sample to a second oligonucleotide array created in step 1 (as described in Example 2); and
5) comparing the resulting hybridization profiles to identify the sequences that are differentially expressed between the first and second samples.
   The subsequently identified genes and related pathways may then be further manipulated in different ways, including, but not limited to, the following:1) they may be used as markers for the particular phenotype induced by the second condition; and 2) they may be manipulated to induce the particular phenotype by the cells in the absence of the correlating second condition. In addition, the regulatory elements of the identified genes and related pathways may be used to generate an expression system, e.g., a ‘stress-inducible’ expression system.

To determine the genes and related pathways involved when CHO cells are grown at a temperature other than the physiological temperature and/or under conditions that promote transgene expression, identical ‘stress’—oligonucleotide arrays were created using the tiling sequences set forth as SEQ ID NOs:3643-7284, i.e., the tiling regions of 1) consensus sequences set forth as SEQ ID NOs:19-3572 generated (see above) from all publicly available hamster sequences and EST sequences isolated from a cDNA library generated with mRNA isolated from CHO cells grown at 37° C. and CHO cells grown at 31° C., 2) transgene sequences set forth as SEQ ID NOs:1-18, 3) other hamster sequences set forth as SEQ ID NOs:3573-3575, and 4) control sequences set forth as SEQ ID NOs:3576-3642 as template sequences for the selection of oligonucleotide probes. The expression of known and previously undiscovered genes by a CHO cell line modified with soluble IL-13 receptor (cell line A), BDD-FVIII-transfected CHO cells (cell line B) and nontransfected control CHO cells (control cell line), each cell line having a first sample of cells grown at 37° C., and a second sample of cells grown at 31° C., was determined. Each culture was run in triplicate or quadruplicate and, as described in Example 2, the total RNA from the first and second samples of each cell line were separately isolated, processed, and hybridized to a created oligonucleotide array. The resulting hybridization profiles were compared, and 31° C.—inducible genes, i.e., the genes present in each second sample that demonstrate at least a two-fold increase in expression level compared to genes in the first sample (for each of the cell lines) were analyzed further and compared for similarities.

Most of the differentially expressed sequences were unique for each cell line (cell line A=59 sequences; cell line B=149 sequences, control cell line=60 sequences), although several expressed sequences (10 sequences) were shared among all three cell lines. Of interest were the sequences that were expressed differentially in both sIL-13r-transfected CHO cells and BDD-FVII-transfected CHO cells cultured at 31° C., when respectively compared to sIL-13r-transfected and BDD-FVII-transfected CHO cells cultured at 37° C. (49 sequences). The 10 genes identified as differentially expressed in all three cell lines when cultured at 31° C. compared to when cultured at 37° C. and the 49 genes identified as differentially expressed in both transfected cell lines when cultured at 31° C. compared to when cultured at 37° C. may be involved in and/or contribute to the increased cellular productivity observed at 31° C., and therefore, could be targets for cell line engineering or as a tool to screen and predict cell lines that will respond favorably to lower temperature culture conditions.

Using the above-described methods and culturing cells under a variety of different culture conditions, the downregulation of expression of 152 individual sequences listed in Table 2 was determined to correlate with growth of the cells in at least one culture condition that promotes cell survival under stressful conditions and/or transgene expression (e.g., culture at a low temperature, culture in the presence of ammonia, culture in highly enriched media, culture with decreased frequency of passaging the cells, etc.). The downregulation of one or more of these genes also correlated with decreased expression of the sialic acid N-glycolylneuraminic acid (NGNA), a potential human antigen. Listed in Table 4 and set forth as SEQ ID NOs:3421-3572 are the genes that are downregulated by transgene-modified cells (and the fold difference of such downregulation) when they are grown at 31° C. compared to when they are grown at 37° C. (Low Temp Data Set), when they are grown in the presence of an additional 40 mM ammonia (NH₄) compared to when they are grown in no additional ammonia (Ammonia Adapted Data Set), when they are grown in highly enriched media for fed batch culture compared to when the cells are grown in media for maintenance cell culture (Fed Batch Adapted Data Set), when they are passaged every 7 days rather than every 3 to 4 days (Extended Culture Adapted Data Set), or when the cells produce less N-glycolyneuraminic acid (NGNA) compared to similar cells that produce more NGNA (NGNA Data Levels Set). Of these sequences, 134 were determined to be previously undiscovered, i.e., novel, in that they have no homology to any known sequences (i.e., have not been sequenced) and/or in that they have not, until now, been shown to be expressed in CHO cells. These sequences are set forth as SEQ ID NOs:3439-3572. In addition, the expression by CHO cells of other novel genes (e.g., Caspase 8 set forth as SEQ ID NO:3573) was also verified.

The above examples demonstrate the use of an oligonucleotide array created according to the methods set forth in Example 1 to verify expression of transgenes by CHO cells to and identify genes potentially involved in transgene expression. The identified genes represent previously undiscovered genes and/or known genes, predicted genes, or novel ESTs, that were previously unknown to be involved in the induction of transgene expression. Thus they provide novel targets that may be manipulated to increase the production of a transgene.

Whereas the above examples demonstrate the present invention utilizing the CHO cell line, it should be apparent to one of skill in the art that the present invention is not limited to use with the CHO cell line. One of skill in the art will know that the examples mentioned above will need only slight modifications to make and use an oligonucleotide array to monitor the expression of genes by bacterial, plant, fungal, and animal cell lines. For example, if it is desired to monitor the known and previously undiscovered genes of a bacterial cell line derived from Staphylococcus aureus, one of skill in the art will know that all publicly available coding sequences from all Staphylococcus aureus strains may be clustered and aligned to identify consensus sequences and, subsequently, to make an oligonucleotide array to known and previously undiscovered genes of Staphylococcus aureus, (in a manner similar to Example 1). Furthermore, without undue experimentation, one of skill in the art will be able to modify the protocols described in Example 2 to make them more appropriate for the cell line that is being analyzed. For example, different protocols are required to isolate RNA from bacterial, plant, fungal, and animal cell lines, and the differences in these protocols are well known in the art. It should also be apparent to one of skill in the art that transgene sequences are not limited to those listed in Table 1. Finally, one of skill in the art will also be able, without undue experimentation, to use an oligonucleotide array created as described herein not only to detect and improve the expression of a transgene, but also to quantify, and enhance the quality of, transgene expression. Consequently, the present invention is not limited to the Examples described above, and can be used to make an oligonucleotide array that can be used to optimize the culture conditions of, and/or transgene expression by, any cell line.

What is claimed:

1. A method of forming an oligonucleotide array directed toward an unsequenced organism, the method comprising the steps of:
(a) identifying a plurality of template sequences, wherein the plurality comprises at least one consensus sequence for a gene expressed by a cell derived from the unsequenced organism; and
(b) selecting a plurality of oligonucleotide probes, wherein the plurality of oligonucleotide probes comprises a first set of oligonucleotide probes, each of which is specific for one of the plurality of template sequences, and wherein at least one oligonucleotide probe is specific for the at least one consensus sequence for a gene expressed by the unsequenced organism,
wherein the step of selecting the plurality of oligonucleotide probes forms the array of nucleic acids.

2. The method of claim 1, wherein the at least one consensus sequence is generated from at least two nucleic acid sequences of cells derived from different genera of the unsequenced organism.

3. The method of claim 1, wherein the at least one consensus sequence is generated from at least two nucleic acid sequences of cells derived from different species of the unsequenced organism.

4. The method of claim 3, wherein the unsequenced organism is hamster.

5. The method of claim 4, wherein the plurality of template sequences further comprises a template sequence selected from the group consisting of the polynucleotide sequences of SEQ ID NOs:19-3575, SEQ ID NOs:3661-7217, complements thereof, and subsequences thereof.

6. The method of claim 5, wherein the plurality of template sequences further comprises at least one transgene sequence.

7. The method of claim 6, wherein the at least one transgene sequence comprises a polynucleotide sequence selected from the group consisting of the polynucleotide sequences of SEQ ID NOs:1-18, SEQ ID NOs:3643-3660, complements thereof, and subsequences thereof.

8. The method of claim 7, wherein the plurality of template sequences further comprises at least one control sequence.

9. The method of claim 8, wherein the at least one control sequence comprises a polynucleotide sequence selected from the group consisting of the polynucleotide sequences of SEQ ID NOs:3576-3642, SEQ ID NOs:7218-7284, complements thereof, and subsequences thereof.

10. The method of claim 9, wherein the plurality of oligonucleotide probes further comprises a second set of oligonucleotide probes, each of which is a mismatch probe for a different oligonucleotide probe of the first set.

11. The method of claim 10, further comprising, after the step of selecting a plurality of oligonucleotide probes, the step of immobilizing the plurality of oligonucleotide probes to a solid phase support.

12. An oligonucleotide array directed toward an unsequenced organism, the array comprising a first plurality of oligonucleotide probes, each of which is specific to one of a plurality of template sequences, wherein the plurality of template sequences comprises at least one consensus sequence for a gene expressed by a cell derived from the unsequenced organism.

13. The oligonucleotide array of claim 12, wherein the at least one consensus sequence for a gene expressed by an unsequenced organism is generated from at least two nucleic acid sequences from different genera of the unsequenced organism.

14. The oligonucleotide array of claim 12, wherein the at least one consensus sequence for a gene expressed by an unsequenced organism is generated from at least two nucleic acid sequences from different species of the unsequenced organism.

15. The oligonucleotide array of claim 12, wherein the unsequenced organism is hamster.

16. The oligonucleotide array of claim 15, wherein the plurality of template sequences further comprises at least one template sequence selected from the group consisting of the polynucleotide sequences of SEQ ID NOs:19-3575, SEQ ID NOs:3661-7217 complements thereof, and subsequences thereof.

17. The oligonucleotide array of claim 16, wherein the plurality of template sequences further comprises at least one transgene sequence.

18. The oligonucleotide array of claim 17, wherein the at least one transgene sequence comprises a polynucleotide sequence selected from the group consisting of the polynucleotide sequences of SEQ ID NOs:1-18, SEQ ID NOs:3643-3660, complements thereof, and subsequences thereof.

19. The oligonucleotide array of claim 18, wherein the plurality of template sequences further comprises at least one control sequence.

20. The oligonucleotide array of claim 19, wherein the at least one control sequence comprises a polynucleotide sequence selected from the group consisting of the polynucleotide sequences of SEQ ID NOs:3576-3642, SEQ ID NOs:7218-7284, complements thereof, and subsequences thereof.

21. The oligonucleotide array of claim 20, wherein the array further comprises a second plurality of oligonucleotide probes, each of which is a mismatch probe for a different oligonucleotide probe of the first plurality.

22. A method for detecting the presence, absence, and/or quantity of expression levels of a plurality of genes in a cell derived from an unsequenced organism comprising the steps of:
(a) forming a hybridization profile by incubating target nucleic acids prepared from the cell with an oligonucleotide array made according to the method as in claim 1, or with an oligonucleotide array as in claim 12; and
(b) detecting the hybridization profile,
wherein the hybridization profile is indicative of the absence, presence and/or quantity of expression levels of a plurality of genes in the cell.

23. The method of claim 22, wherein the plurality of genes comprises at least one previously undiscovered gene of the cell.

24. The method of claim 22, wherein the plurality of genes comprises at least one transgene.

25. The method of claim 23, wherein the cell is derived from hamster.

26. The method of claim 25, wherein the cell is a CHO cell.

27. The method of claim 24, wherein the cell is derived from hamster.

28. The method of claim 27, wherein the cell is a CHO cell.

29. A method for comparing expression levels of a plurality of genes in a first cell derived from an unsequenced organism to expression levels of the plurality of genes in a second cell derived from the unsequenced organism, the method comprising the steps of:
(a) forming a first and a second hybridization profile, wherein the first hybridization profile is formed by incubating target nucleic acids prepared from the first cell with a first oligonucleotide array made according to the method as in claim 1, or with a first oligonucleotide array as in claim 12, and wherein the second hybridization profile is formed by incubating target nucleic acids prepared from the second cell with a second array identical to the first array;
(b) detecting the first and the second hybridization profiles; and
(c) comparing the first and second hybridization profiles.

30. The method of claim 29, wherein the first cell and the second cell are from the same cell line, wherein the first cell is modified with a transgene, and wherein the second cell is not modified with a transgene.

31. The method of claim 29, wherein the first cell differs from the second cell with respect to a culture condition.

32. The method of claim 31, wherein is the culture condition is selected from the group consisting of duration of culture, temperature, serum concentration, nutrient concentration, metabolite concentration, pH, lactate concentration, ammonia concentration, oxidation level, sodium butyrate concentration, valeric acid concentration, hexamethylene bisacetamide concentration, cell concentration, cell viability, and recombinant protein concentration.
